(19) United States
(12) Patent Application Publication
(10) Pub. No.: US 2008/0269871 A1
(43) Pub. Date: Oct. 30, 2008

(54) Implantable device with miniature rotating portion and uses thereof

(76) Inventor: Uri Eli, Kiryat Ono (IL)

Correspondence Address: Dr. D. Graeser Ltd.;c/o The Discovery Dispatch, 9003 Florin Way, Upper Marlboro, MD, 20772, US

(21) Appl. No.: 11/976,436

(22) Filed: Oct. 24, 2007

Related U.S. Application Data
(60) Provisional application No. 60/924,041, filed on Apr. 27, 2007.

(51) Int. Cl. A61F 2/82; A61M 29/00

(52) U.S. Cl. 623/1.15; 606/200; 623/1.42

(57) ABSTRACT

A miniature rotating portion, used within the human body. Optionally and preferably, the device may be used for one or more of active filtration and removal of emboli, plaques and occluding materials within the cardiovascular system and any other bodily passageway, promoting blood clots and occluding material lysis and disassembly.

This Application claims priority from U.S. Provisional Application No. 60/924,041, filed on Apr. 27, 2007, which is hereby incorporated by reference as if fully set forth herein.

### FIELD OF THE INVENTION

The present invention relates to an implantable device with at least one miniature rotating portion and optionally at least one filtering portion and the various uses and methods thereof, and in particular to such devices for use in the human body.

### BACKGROUND OF THE INVENTION

One of the leading causes of death is cardiovascular disease particularly due to blockage of blood vessels. Such blockage may lead to death by stroke or heart attacks. In order to diagnose and monitor the disease, many devices, drugs, therapies and treatments have been developed to visualize, diagnose and prevent the onset and deterioration of cardiovascular disease. Currently, one of the only small scale devices used in treating cardiovascular disease is the stent. Current cardiovascular imagery technology includes: duplex ultrasound, color-flow imaging, Magnetic Resonance Angiogram (MRA), Computerized Tomography Angiogram (CTA) and Laser Doppler Imagery.

Cardiovascular imagery devices described above may be used to visualize vessels; however, although they may identify a diseased vessel they do not prevent blockage of vessels in disease states. Once a blocked vessel has been identified it is commonly treated by implanting a stent.

Stents are well known devices for placement in vessels of the human body to obtain and maintain patency of that vessel. The greatest use for stents has been for placement within a stenosis in a coronary artery.

Recent improvements in the design of stent delivery systems have made it possible to eliminate the step of pre-dilatation for the treatment of many classes of stenoses. The delivery of a stent to the site of a stenosis without pre-dilatation has been given the name “direct stenting”.

Stents are particularly useful in the treatment of atherosclerotic stenosis in arteries and blood vessels. An intravascular interventional device such as a stent is particularly useful in the treatment and repair of blood vessels after a stenosis has been treated by percutaneous transluminal coronary angioplasty (PTCA), percutaneous transluminal angioplasty (PTA), or removed by atherectomy or other means, to help improve the results of the procedure and reduce the possibility of restenosis. Stents also can be used to provide primary compression to stenosis in cases in which no initial PTCA or PTA procedure is performed. While stents are most often used in the procedures mentioned above, they also can be implanted in any body lumen or vessel such as the urethra, esophagus and bile duct and the like.

In typical PTCA procedures, a guiding catheter or sheath is percutaneously introduced into the cardiovascular system of a patient through the femoral arteries and advanced through the vasculature until the distal end of the guiding catheter is in the aorta. A guide wire and a dilatation catheter having a balloon on the distal end are introduced through the guiding catheter with the guide wire sliding within the dilatation catheter. The guide wire is first advanced out of the guiding catheter into the patient's vasculature and is directed across the arterial lesion. The dilatation catheter is subsequently advanced over the previously advanced guide wire until the dilatation balloon is properly positioned across the arterial lesion. Once in position across the lesion, the expandable balloon is inflated to a predetermined size with a radio-opaque liquid at relatively high pressure to press the atherosclerotic plaque of the lesion against the inside of the artery wall and thereby dilate the lumen of the artery. The balloon is then deflated to a small profile so that the dilatation catheter can be withdrawn from the patient's vasculature and the blood flow resumed through the dilated artery.

In angioplasty procedures of the kind referenced above, abrupt re-closure may occur or restenosis of the artery may develop over time, which may require another angioplasty procedure, a surgical bypass operation, or some other method of repairing or strengthening the area. To reduce the likelihood of the occurrence of abrupt re-closure and to strengthen the area, a physician can implant a stent inside the artery across the lesion.

Stents are usually delivered in a compressed condition to the target location and then are deployed into an expanded condition to support the vessel and help maintain it in an open position. The stent is usually crimped tightly onto a delivery catheter and transported in its delivery diameter through the patient's vasculature. The stent is expandable upon application of a controlled force, often through the inflation of the balloon portion of the delivery catheter, which expands the compressed stent to a larger diameter to be left in place within the artery at the target location. The stent also may be of the self-expanding type formed from, for example, shape memory metals or super-elastic nickel-titanium (NiTi) alloys, which will automatically expand from a compressed state when the stent is advanced out of the distal end of the delivery catheter into the body lumen. Most notably, upon stent deployment and expansion, the stent is stationary and cannot be relocated even in case of stent drift or incorrect stent positioning during deployment. To circumvent a misguided stent the preferred solution is to implant a second stent instead of the shifted stent, however, this increases the probability for stent related complications.

The above-described, non-surgical interventional procedures, when successful, avoid the necessity for major surgical operations. However, a danger which is always present during these procedures is the potential for particles of the atherosclerotic plaque, which can be extremely friable, to break away from the arterial wall. For example, during deployment of a stent, the metal struts of the stent can possibly cut into the stenosis and shear off pieces of plaque which become embolic debris that will travel downstream and lodge somewhere in the patient's vascular system.

The above danger is particularly acute when any of the above-described procedures are performed in the carotid arteries, the release of emboli into the circulatory system should be avoided. For example, debris that is carried by the bloodstream to distal vessels of the brain can cause these cerebral vessels to occlude, possibly resulting in a stroke. Therefore, although cerebral percutaneous transluminal angioplasty has been performed in the past, the number of procedures performed has been limited due to the fear of causing an embolic stroke should embolic debris enter the bloodstream and block vital downstream blood passages. Embolization in other parts of the vasculature may induce possible acute myocardial infarction when a procedure is performed on the coronary arteries and gangrene when performed in peripheral arteries, such as the arms and legs.

While stents are helpful in holding open otherwise blocked or occluded vessels, their structure permits such material to enter the bloodstream from the vessel walls. Due to the open nature of the stent structure, there is a possibility that growth material can pass through the openings between the struts and extend into the inner lumen of the stent structure. For example, excessive cell or tissue growth (intimal hyperplasia) can cause partial restenosis to develop over time, which is detrimental to the patient. The tissue or cell growth can extend into the tubular opening created by the stent and can block or otherwise re-occlude the opening and can possibly cause abnormal blood flow through the stent which can cause formation of thrombi that are detrimental to the patient's health.

Background art devices have been created to help reduce the passage of such growth through the wall of the deployed stent, including a stent covering which surrounds the open stent. In this manner, the gaps between the stent struts can be covered to prevent material, such as plaque, from prolapsing between the struts. Coverings have included a variety of materials such as ePTFE, autologous vein grafts, pericardium and fibrin. The covering should be sufficiently flexible and expandable to allow the stent to deploy from its collapsed or compressed position to a fully expanded position.

Covered stents also help prevent the struts from cutting into the plaque of the stenosis which helps reduce the possibility of forming embolic debris that can be released into the blood stream, as described above. Moreover, in the event that any embolic debris is created from the expansion of the stent, the covering could trap the embolic particles against the arterial wall, thus preventing the particles from being released into the bloodstream.

Such covered stents are difficult to manufacture due to the flexibility of the covering and the requirement that the covering be capable of expansion when the stent is deployed within the patient's vasculature. For these reasons, the material used to form the covering may be subjected to intricate processing to obtain the desired flexibility for the covering and to attach the covering to the stent. A covering which does not expand normally can cause the stent to misalign within the body vessel and can cause a non-uniform deployment of the stent. Moreover, some coverings are made from a sheet of material which is rolled into a cylindrical shape by creating a longitudinal seam which runs along the length of the covering and then the covering is attached to the stent. Such coverings can be more susceptible to tearing, especially at the seam, when the stent is expanded.

Some stents that are covered may have a tendency to shorten when expanded and the covered material also shortens, providing an undesirable result. As the stent and the covered material are expanded into contact with an artery or vessel wall, the shortening movement may scrape along the artery wall and cause injury or dislodge plaque material which may cause embolisms. Further, as these covered stents shorten upon expansion, the cover material shortens past the stent struts at the stent ends, leaving a covered stent with exposed stent struts, and not fully covered upon expansion.

Despite the vast use of stents in medical procedures and the many types of stent structures available today, multiple studies have suggested the risk of blood clots, heart attack and death rises with drug-coated stent in patients that stop taking Plavix® and Aspirin® early on after implantation. The data revealed that drug coated stents cause a 0.4 to 0.6 percent risk of blood clot formation (depending on the stent manufacturer). Furthermore, it has been documented that stent-related blood clots, formed and released into the blood circulation, following the deployment of a drug eluted stent, have a 45% probability of causing a major heart attack that leads to death. This is due to the fact that blood clots that form in the body may migrate through the blood vessels and may cause a major heart attack, stroke or shock (when reaching to the lungs).

Thrombus formation, and subsequent movement to form an embolus, may also occur in the heart or other parts of the vascular system, causing acute reduction of blood supply and hence ischemia. The ischemic damage often leads to tissue necrosis of organs such as the kidneys, retina, bowel, heart, limbs, brain or other organs, or even death. For example, blood clots may form in the veins of subjects that are immobilized, particularly in the legs of bedridden or otherwise immobilized patients. These clots may then travel in the bloodstream, potentially to the arteries of the lungs, leading to a common, often-deadly disease called ‘pulmonary embolus’ (PE).

Clots or thrombi that become dislodged from their point of origin are termed emboli. Vascular emboli are the major single causative agent for multiple human pathologies. It is a leading cause of disability and death.

Vascular emboli may originate from a blood clot occurring in the heart, but may also be derived from body tissues. For example, atherosclerosis, or hardening of the blood vessels from fatty and calcified deposits, may cause particulate emboli to form. Moreover, clots can form on the luminal surface of the atheroma, as platelets, fibrin, red blood cells and activated clotting factors may adhere to the surface of blood vessels to form a clot. Various types of filters have been proposed in an attempt to remove or divert such particles from the bloodstream before they can cause damage to bodily tissues.

Many transvenous filtering devices have been developed for installation in the inferior vena cava preventing especially large clots, caused by Deep Vein Thrombosis (DVT), from migrating from the leg veins to the lungs. These filters have fine wires positioned in the blood flow to catch and hold clots for effective lysis in the blood stream. In most cases the filter wires are formed from nitinol (nickel-titanium alloy). Nitinol is a plastically deformable material having temperature-sensitive shape memory with a transition temperature around body temperature. Alternatively, filters may be formed from elastic material having a core formed from radio opaque material. Such filters are passive and do not act in any form to accelerate the breakdown and lysis process of the blood clot.

For example, U.S. Pat. No. 6,258,120 discloses a filter device intended to be inserted into the artery of a patient. However, the device has an inherent drawback, which is that the actual trapping of an embolus, for successful operation of the device, may result in blockage of blood flow through the device and hence through the artery. Other disclosed embodiments of the device, which may not be blocked by clots, are not able to filter clots, and may in fact funnel such particulate matter to the blood vessels leading to the brain. None of the disclosed embodiments of the device are anchored to the artery, but instead rely upon conformation to the arterial shape and size to maintain the position of the device, which is not secure.

U.S. Pat. Nos. 4,873,978; 5,814,064; 5,800,457; 5,769,816; and 5,827,324 describe devices that are intended only for temporary insertion into a blood vessel. Therefore, these devices avoid the difficult issue of simultaneously successfully filtering emboli while also maintaining blood flow through the blood vessel. As such, they do not address the problem of prolonged filtration of the blood.

### SUMMARY OF THE INVENTION

There is an unmet need for, and it would be highly useful to have, a device that is able to protect a blood vessel and body tissues against damage caused by particulates such as an embolus.

The present invention overcomes these drawbacks of the background art by providing a device, system and method of use thereof for protecting a blood vessel and/or body tissues against damage or occlusion caused by particulates in the blood stream, which may optionally be an embolus, a foreign body or any undesirable physical material in the bloodstream, in an active manner, by harnessing a small portion of the freely available hydrokinetic energy inherent in the human body blood flow and converting it into mechanical energy. Blood vessel protection may optionally be gained by capturing and/or disassembling embolic material or occluding material.

According to preferred embodiments, the present invention is also able to treat any existing blockages, prevent new blockage or clot formation, maintain vessel patency, improve the structural integrity of stents and filters, maintain vessel patency of a stented vessel, prevent restenosis, and filter and promote lysis of occluding material such as blood clots.

The different optional embodiments of the present invention provide for a device and method for protecting a blood vessel, and hence bodily tissues, from damage caused by particulate such as an embolus, a foreign body, or body tissues, by removing such particulate material. By “removing” it is meant any type of elimination, including filtering, trapping, reduction of larger particles to smaller (and hence less potentially harmful particles) and the like.

According to some embodiments there is provided a device comprising a vessel support structure, at least one rotating portion, and at least one filtering portion, wherein the rotating and filtering portions are preferably supported and maintained by the vessel support structure. Optionally, the rotating portion may be used independently of the vessel support structure or the filtering portion. Optionally, these components may optionally be integrated within one another producing a single unit comprising at least two of these components. Optionally each of the components may be differentially attached or coupled to one another forming any number of combinations. For example, one such formation may optionally comprise a vessel support structure coupled to a rotating portion having a filter portion incorporated internally. Optionally, a filter portion may be shaped to encase at least a part of a rotating portion. Optionally, a filter portion shaped to span a section of a vessel therefore behaves as a vessel support structure optionally having a rotating portion attached thereto.

Optionally the rotating portion and filtering portion may be integrally formed within the vessel support structure. The rotating portion and filtering portion may be optionally attached or coupled thereto either during manufacture, or after implanting the vessel supporting structure within a blood vessel or any other bodily passageway.

The vessel support structure and the rotating portion of the device are optionally and preferably made of a dilatable and/or otherwise self-expanding structure that is optionally shaped according to the vessel, such as but not limited to a tubular structure, stent like structure, off the shelf (OTS) stent or filter structure, or wire frame or other like vessel support structure. Similarly any of the embodiments of the present invention may be optionally implemented as a Stent-Graft that is optionally implanted in the aorta preferably in place of surgery. Various optional and preferable embodiments, use variation and implementations of this basic structure presented herewith.

The present invention overcomes the disadvantages of the background art in many ways, including but not limited to by providing a device comprising at least one miniature rotating portion, optionally anchored to a vessel support structure used within the cardiovascular system. Optionally the device of the present invention may be implanted within the cardiovascular system, for example, in vessels such as the aorta, arteries, veins or any blood vessel or other bodily passageways having a flowing fluid. Optionally and preferably, the device may be used for active filtration and removal of plaques in the blood vessel thereby providing a means for accelerating plaque breakdown and lysis.

Any of the embodiments of the present invention may be deployed to a blood vessel. The deployment site may be optionally and preferably determined by any number of imaging methods known in the art and incorporated herein by reference for example including but not limited to X-ray fluoroscopy, intravascular ultrasound, echocardiography, MRI (magnetic resonance imaging), angioscopy, CT (computerized tomography) scan, and/or any other suitable imaging technology. Another optional mode of deployment is surgical, by direct insertion of the catheter carrying the device through a puncture of the targeted vessel in proximity to the deployment site.

Vessel support structures such as stents are implanted within blood vessels and are therefore subject to and accommodate blood flow and circulation. Blood circulates in the human body for the same reason that any fluid flows, due to a pressure gradient that drives the fluid from an area of high pressure to an area of relatively lower pressure. In the human body, blood circulates by the pumping action of the heart in a repetitive predictable manner, from the ventricles returning to the atria, due to the blood pressure gradient that exists between them. When the heart beats, the pressure in the arteries increases to a maximum, thereby creating the systolic pressure gradient that drives blood through the vessels. It is this pressure gradient, manifested in the blood stream that creates the hydrokinetic energy source that may be harnessed by the preferred embodiment of the present invention. Similarly, during lower diastolic blood pressure, the rotating portion of the preferred embodiment of the present invention preferably continues to work such that its function is not limited by the level of pressure gradient. Furthermore, an optional embodiment of the present invention is able to continue working for a period of at least a few seconds with zero blood flow due to the momentum of the rotating portion blades. This preferred embodiment enables the rotating portion to work continuously regardless of the blood pressure phase.

A preferred non limiting embodiment of the present invention improves the use of vessel support structures, including but not limited to a tubular structure, stent, blood clot filter, wire frame and stent-graft, by introducing at least one miniature device, preferably in the form of a hydrokinetic rotating portion. Optionally and preferably the rotating portion reduces the likelihood of reformation of blockage in a blood vessel following stent implantation, and filters and removes plaque and embolic material in the bloodstream.

The rotating portion according to a preferred non limiting embodiment of the present invention is able to harness the hydrokinetic energy of blood flow that causes the rotating portion blades to spin. The rotating portion of the preferred embodiment is preferably stably connected to a vessel support structure for example including but not limited to a tubular structure, a stent, blood clot filter, wire frame or stent-graft or the like. Optionally, the rotating portion is stably connected to the frame of a vessel support structure in a number of optional configurations preferably including but not limited to radial placement within the vessel support structure, wherein the rotation portion is within the lumen of the vessel support structure. Optionally, the rotating portion may be coupled to the frame of the vessel structure wherein the rotating portion is optionally located extra-luminally, outside the lumen of the vessel support structure. Optionally, the rotating portion may be integrated within the plane of the vessel support structure's frame, preferably continuously integrated within the frame of the support structure. Optionally, the rotating portion may be not be connected to the frame of a vessel support structure and may instead optionally freely rotate within the vessel support structure, preferably encased within the vessel support structure. The rotating portion's blades optionally and preferably function to clear the vessel support structure and its vicinity of any blockages or emboli that may have formed while optionally preventing any new blockages or emboli from reforming.

A preferred non limiting embodiment of the present invention comprises a vessel supporting structure and a rotating portion. The vessel support structure may be optionally composed of metal or plastic or any combination thereof serving as the mounting platform for the rotating portion. Optionally the vessel support structure may feature one or more existing Commercial Off The Shelf (COTS) stents, blood clot filters or any other implanted devices.

The vessel support structure and the rotating portion according to the preferred exemplary embodiment of the present invention is preferably expandable from a low-profile compressed condition to a larger profile expanded condition, wherein the resilient material induces the vessel supporting structure to expand radially, and to thereby apply radial force against the blood vessel's inner wall surface.

The preferred embodiments of the present invention comprising the vessel support structure and the rotating portion may preferably be composed of a shape memory alloy (SMA), including but not limited to nickel titanium alloy (NiTi), also known as nitinol, having transition temperature around body temperature; or optionally a shape memory polymer (SMP) that can be triggered in response to changes in heat, pH, electric or magnetic fields; or a combination thereof. For example, the device of the preferred embodiment of the present invention is optionally and preferably introduced into a blood vessel in its collapsed formation having a small profile, featuring a relatively narrow diameter. Once in place and after it is released from the constraining catheter, the device preferably expands to the appropriate diameter and into its final or “memorized” shape.

Optionally and preferably the rotating portion, according to a preferred embodiment of the present invention, is capable of axial flow as an axial flow rotating portion. More preferably, the rotating portion is an across-flow rotating portion. Of course any type of rotational direction may optionally be implemented. Such a rotating portion is preferably associated with the vessel support structure and is preferably smaller than the diameter of the vessel support structure's lumen. Optionally at least one rotating portion may be present within the diameter of the lumen. Optionally and preferably a plurality of rotating portions may be placed sequentially and incrementally, in a step like manner, to diagonally span the diameter of the vessel support structure. Optionally a plurality of rotating portions may so be placed wherein any cross section angle, including horizontally or vertically, is covered.

The optional, exemplary cross flow rotating portion preferably comprises a plurality of blades that are optionally shaped like a helical or skewback airfoils. These blades are optionally and preferably oriented transversely and perpendicularly to the fluid flow and parallel to the axis of rotation. Optionally, the blades have hydrofoil sections that provide tangential pulling forces in the cross fluid flow, allowing the forces to rotate the rotating portion in the direction of the leading edge of the blades. Therefore the direction of rotation of the rotating portion preferably depends significantly, and more preferably only, on blade orientation, rather than on the direction of fluid flow.

Preferably the blades of this embodiment of the present invention are helical airfoils that are warped into a spherical or elliptic shape. Optionally, the blades may have variable widths along the blade and/or its shaft. Optionally, different combination of blade types may be used an individual rotation portion. The orientation and shape of the rotating portion's blades preferably allow the rotating portion to be self-starting rotating portion, such that rotation is preferably initiated upon initiation of blood flow and such that the blades rotate even in very slow blood flow. Optionally, the shape and dimensions of the rotating portion may be controlled and adjusted to best accommodate one or more of vessel geometry and flow rate (and pressure). Optionally, the various components comprising the rotating portion, for example preferably including the blades and anchors, may be controlled and adjusted to best accommodate one or more of vessel geometry and flow rate (and pressure).

Optionally, the rotation and speed of the rotating portion may be controlled. The rotational spin of the rotating portion may optionally be gained from or induced by external sources (other than blood flow), for example including but not limited to magnetic energy induction. The blades of the rotating portion structure according to this optional embodiment may optionally comprise magnetic material. Optionally the blades may be composed of and/or integrate and/or otherwise feature permanent magnets. Alternatively, the blades could be produced from and/or coated with magnetic material.

In another embodiment, the rotating portion control is accomplished by placing one or more endoluminal electrical cables connected to conductive 1o windings, which may optionally be incorporated within a vessel supporting structure. This structure would induce rotation of the rotating portion in a similar manner to the rotation of electrical engine. Furthermore, according to this optional embodiment, such control and stimulus from external sources may be used to recover or “restart” the operation of the rotating portion in case of interference, for example if tissue build-up interferes with the rotating motion.

It should be noted that the rotating portion preferably harvests the hydrokinetic energy of the rotating portion sectional area through which the fluid passes. The shape, properties and number of blades of the rotating portion of the preferred non limiting embodiment of the present invention optionally provides the rotating portion with efficiency control.

One of the many distinct advantages of such a preferred embodiment of the present invention is the ability to control the rotating portion's parameters. Optionally, control of the rotating portion may be gained by adjusting one or more of the blades' properties, for example including but not limited to blade shape, length, width, material or the like. This allows the rotating portion to be customized to have specific rotational efficiency for allowing the fluid to flow freely. Such free fluid flow prevents interference with blood flow and reduces or even eliminates damage to blood cells; it also enables the device to be adjusted to best suit the vessel shape.

The blades are optionally mounted with at least one supporting member, which may optionally be mounted onto a rotatable shaft or a fixed shaft supported by at least one lightweight structure. The blades attached to an axis or shaft produce rotational spin about the axis creating rotational mechanical energy that may optionally be used to clear the lumen of the vessel support structure of any accumulated plaque, emboli or occluding material. Optionally, the blades and supporting members may rotate freely in the blood stream within a vessel support structure but without an axis or shaft.

According to an optional embodiment, the device further comprises a flexible shaft, wherein the blade is supported by the shaft and the shaft is connected to the support structure, more preferably wherein a plurality of anchors are connected to the flexible shaft.

The blades of the rotating portion structure according to this preferred embodiment may optionally be composed of and/or integrate permanent magnets on their external surface, and/or optionally the blades could be produced of or coated with magnetic material.

Optionally, the rotating portion according to any one of the embodiments of the present invention may comprise at least two or more blades for example including three, four or five blade configurations.

The vessel support structure, the rotating portion and the filter portion in accordance with a further optional preferred embodiment of the present invention may produce electrical energy (electric potential), or electrostatic energy by the addition of metals and/or polymers that are naturally charged, or, alternately, by incorporating piezo-electric materials which may generate electric potential. Such metals, polymers and piezo-electric materials may generate electricity or electric potential by the rotating action of the rotation device that generated electric potential.

According to some embodiments, the present invention is an inherent blood flow rate sensor, in which the blood flow rate passing through the device is proportional to the rotational speed of the rotating portion.

Some embodiments of the present invention provide for a device and method for protecting by filtering a cardiovascular vessel, and hence bodily tissues, against damage caused by particulates such as an embolus, foreign matter, and body tissues.

In another general aspect, according to some embodiments the invention features an active filtering device where a filter portion is incorporated with a rotating portion system in order to enhance the capturing and lysis capability of the device of the present invention. Optionally the blood clot filtering portion is deployed between the rotating portion blades and fixedly coupled to the rotating portion. The filtering portion is preferably formed from a plurality of elongated strands arranged to form a filtering structure, optionally including but not limited to a conical filtering portion and/or a structure resembling a spider's net, to capture and hold the blood clots flowing in the blood stream. Optionally, the filtering portion is attached to the blades, and thereby rotates together with the blades.

The blades' rotation speed is optionally and preferably higher than the velocity of the fluids that passes through the rotating portion. Thus, this higher speed exposes the captured blood clots to higher velocities than the blood flow velocity, and achieves better and effective blood clot lysis. Once implanted, a preferred embodiment of the present invention optionally enables the rotating blades and filter portion to capture and remove the blood clot from the blood flow, by wrapping the blood clots around the rotating portion blades and/or effectively capturing it using the filter portion. The blood clots that are captured by the filter portion experience higher velocity and momentum than the velocity of the blood flow, due to the rotating portion's rotation. This higher velocity actively promotes and accelerates the effective disassembly and lysis action of the blood clot.

The occluding body preferably causes a change in the rotational of the blades, either directly by associating with the blades or indirectly by affecting blood flow rate. The change in the rotating portion activity and motion of a preferred embodiment of the present invention allows the change to be monitored or detected. Such monitoring or detection may be optionally performed according to imaging methods known in the art for example including but not limited to intravascular ultrasound, MRI (magnetic resonance imaging) or magnetic fluctuation sensor.

A still further optional non limiting embodiment provided by the present invention is an inherent blood flow rate sensor, in which the blood flow rate passing through the device is proportional to the rotational speed of the rotating portion. Optionally, this may serve as a primary or secondary sensor for devices associated with the rotating portion of the preferred embodiment of the present invention including but not limited to a implanted cardioverter defibrillator (ICD), potentially minimizing the defibrillator's false alarm rate. This exemplary non-limiting embodiment may optionally communicate vital data regarding the flow rate, cardiac output and vessel occlusion state as inferred from the activity or inactivity of the rotating portion. Optionally, the activity level of the rotating portion may be visualized by imaging methods known in the art, for example including, but not limited to intravascular ultrasound, MRI (magnetic resonance imaging) or magnetic fluctuation sensor

The filter device of the present invention may optionally be used in the cardiovascular system such as the carotid artery, aorta, veins, or in any blood vessel or in any other bodily passage, optionally having at least one blood entrance inlet, and at least one blood exit outlet, and therefore, may be positioned also in the vicinity of a vessel bifurcation or surgical vessel bypass.

Preferably the rotating portion and the filter portion may be fitted within the vessel support structure. Optionally the rotating portion and the filter portion may be fitted on the surface of the vessel support structure in the vicinity of bifurcate junctions, inlets and outlets. The rotation portion may be fitted on the external surface of the vessel support structure, the internal surface of the vessel support structure or fitted within the vessel support structure or any combination thereof.

A preferred non limiting embodiment of the present invention improves the use of a vessel support device including but not limited to a stent, blood clot filter, stent-graft or wire frame, by introducing at least one rotating portion and at least one filtering portion that optionally routes and diverts the flow of embolic material, preferably actively deflecting embolic matter using the rotating portion sending the flow of embolic material to safer areas. More preferably the device captures and disassembles embolic material and occluding material while preventing any new material from reforming, produce electric charge to prevent blood components from collecting on the rotation portion.

A further optional embodiment of the present invention features an inferior vena cava (IVC) blood clot filter with at least one rotating portion and/or filtering portion which may optionally be incorporated with existing off the shelf (OTS) IVC blood clot filters, in order to enhance its capturing and lysis capability. An exemplary embodiment of the present invention comprises a blood clot filtering portion that is optionally deployed between the rotating portion blades and fixed to the rotating portion. The filtering portion is optionally and preferably formed from a plurality of elongated strands arranged to form a generally filtering structure to capture and hold the blood clots flowing in the blood stream. The filtering portion is attached to the rotating portion, and thereby rotates together with the rotating portion.

A still further optional embodiment of the present invention comprises a temporary and retrievable IVC blood clot filter device. Optionally the device of the present invention is implanted in a stationary position for a limited period of time when temporary IVC filter placement is required in case of recurrent PE, surgery or other medical need and is preferably retrieved by medical instrumentation such as but not limited to an introducer catheter that may be re-advanced over the guidewire to a site of the device assembly in order to retrieve the device.

A further embodiment of the present invention is optionally realized by combining the rotating portion and filter portion with existing off the shelf (OTS) IVC filters in order to generate a double-stage filter that combines an active filter, for example including the rotation portion of the present invention and passive filter protection, optionally including but no limited to an IVC filter.

The invention is further directed to an IVC filter optionally positioned in the inferior vena cava and optionally coupled to an additional stage of at least one rotating portion and filtering portion.

While in operation, the rotating portion and filtering portion preferably capture the blood clot flowing in blood. The captured blood clots within the filter portion and/or the rotating portion experience higher velocity than the velocity of the blood flow and momentum force due to the rotations. This increased velocity greatly and actively accelerates the effective disassembly and lysis action of the trapped blood clots.

A still further optional and preferable embodiment of the present invention is an implantable device for positioning in the vicinity of the bifurcation of the common carotid artery (CCA) into the internal carotid artery (ICA) and the external carotid artery (ECA), preferably comprising a vessel supporting structure, a rotating portion and a filtering portion. Preferably the filter device may optionally provide at least one, or more preferably, a combination of preventative and protective measures against embolic/thrombosis material buildup optionally including but not limited to:

1) Routing and diverting the flow of embolic material flowing in the CCA toward the ICA, into the ECA;

2) Deflecting embolic material flowing in the CCA toward the ICA, into the ECA;

3) Capturing, holding and disassembling embolic material flowing in the CCA toward the ICA;

4) Disassembling occluding and thrombi material that may have formed on the device while preventing any new material from reforming;

5) Produce electric charge to prevent blood components from collecting.

An optional aspect of the present invention provides for an implantable device preferably comprising vessel support structure, optionally anchored to the inner walls of the carotid artery or the aorta, at least one rotating portion and at least one filtering portion, preferably for preventing the flow of embolic and thrombi material (blood clots) into the ICA. Preferably the vessel support structure functions without obstructing blood flow into the ICA.

A further embodiment of the present invention relates to a permanent implantable device for chronic conditions or long term treatment or retrievable (temporary) implantable device for acute conditions or procedures. In case of a retrievable device, it is preferably retrieved by medical instrumentation, including but not limited to an introducer catheter that may be re-advanced over the guidewire to a site of the device assembly in order to retrieve the device or one of its components.

The rotating and filtering portions comprised in the device of the present invention are optionally and preferably, positioned at the inlet into the internal carotid artery. Optionally the vessel support structure may be positioned in a variety of locations. Optionally, the rotating and filtering portions may be positioned at any location that preferably fulfill at least the following conditions: maintaining an open lumen that does not occlude the flow of blood into the ICA and the ECA, and preventing the passage into the ICA of embolic material and the formation of thrombi material in the vicinity of the device and the ICA inlet.

For example, the vessel support structure may be optionally positioned in the ICA and protrude into the bifurcation zone and optionally still even further into the CCA. Optionally the vessel support structure may be positioned at the entrance to the ECA and extend toward the surrounding walls and even further into the CCA, preferably to gain constructive and supportive strength.

In another exemplary embodiment, vessel support structure may optionally be positioned somewhere along the ICA or in the vicinity of the ICA inlet without protruding into the bifurcation zone, while the rotating and filtering portions preferably act to prevent the passage of embolic material to the brain and the formation of thrombi and occluding material in the vicinity of the device. Optionally and alternatively the vessel support structure may be positioned at the aorta preventing the passage of embolic material from the heart to the brain via the aorta and the formation of thrombi and occluding material in the vicinity of the device

In accordance with an optional embodiment of the present invention, vessel support structure is optionally positioned within the CCA without protruding into the bifurcation zone, wherein the vessel support structure optionally and preferably encase at least one rotating portion and at least one filtering portion.

In accordance with a further optional embodiment of the present invention, a vessel support structure is preferably positioned within the CCA with an upstream portion extending towards the bifurcation zone, wherein the upstream portion preferably accommodates at least one rotating portion and at least one filtering portion.

In accordance with still another preferred but optional embodiment of the present invention, vessel support structure is preferably positioned within the CCA with an upstream portion extending towards the bifurcation zone, wherein the upstream and/or downstream portions (within the CCA) optionally and preferably accommodate at least one rotating portion and at least one filtering portion. Preferably this not only prevents the passage of blood clots into the ICA but also preferably prevents the passage of clots into the ECA.

In accordance with a still further preferred embodiment of the invention, vessel support structure is preferably positioned within the ICA in the vicinity of the entrance to the ICA with a downstream portion adjacent to the bifurcation zone, but without protruding into the bifurcation zone, wherein the downstream portion optionally and preferably accommodate at least one rotating portion and at least one filtering portion.

In accordance with a still further optional and preferred embodiment of the present invention, the vessel support structure is preferably positioned within the ICA but adjacent to the bifurcation zone, wherein the vessel supporting structure comprises at least one rotating portion and at least one filtering portion.

In accordance with a still further optional and preferred embodiment of the present invention, a vessel support structure is preferably positioned within the ECA, optionally with a downstream portion extending towards the bifurcation zone, wherein the downstream portion accommodate at least one rotating portion and at least one filtering portion.

In accordance with a still further optional and preferred embodiment of the present invention, a vessel support structure is preferably positioned within the ECA, with a downstream portion extending towards the bifurcation zone, wherein the downstream and upstream portions accommodate at least one rotating portion and at least one filtering portion. Preferably this formation not only preventing the passage of debris into the ICA of but it also prevents its passage into the ECA.

It will also be appreciated that optionally the vessel supporting structure is positioned within a vascular portion extending along the CCA and the ECA, wherein at least one rotating portion and filtering portion are preferably situated at the inlet to the ICA.

The vessel support structure is optionally and preferably positioned within a vascular portion extending along the CCA and the ICA, wherein at least one rotating portion and filtering portion are preferably situated within the vessel supporting structure while at least one rotating portion and filtering portion is preferably situated at the inlet to the ECA. Preferably, this not only prevents the passage of blood clots into the ICA but also prevents such passage into the ECA.

A still further optional embodiment of the present invention features the vessel support structure being preferably positioned optionally at the bifurcation zone or at the ICA vessel or at the CCA vessel, while at least one rotating portion and filtering portion are optionally situated within the vessel supporting structure or adjacent the inlet into the ICA.

Another optional embodiment of the present invention provides an implantable device optionally for implanting at the vicinity of bifurcation of the common carotid artery (CCA) optionally into the ICA or ECA or at the ICA or at the CCA; optionally the device comprises a vessel supporting structure positioned to the inner walls of a carotid artery, and at least one rotating and filtering portion, wherein at least one rotating and filtering portion is so positioned and sized so that embolic material is prevented from continuing to flow into at least the ICA and most preferred into the ICA and ECA.

A further optional embodiment of the present invention provides an implantable device for implanting at the vicinity of bifurcation of the common carotid artery (CCA) optionally into the ICA and ECA or at the ICA or at the CCA. Optionally the device comprises a vessel supporting structure preferably positioned to the inner walls of a carotid artery, at least one rotating portion and at least one filtering portion, wherein at least one rotating portion and filtering portion is preferably positioned and sized so that occluding and thrombic material that may obstruct blood flow or flow into the ICA is prevented from forming. Most preferably any occluding and thrombic material in the vicinity of the device along the CCA and into the ICA and ECA is prevented from forming. Preferably in the case that some occluding and thrombic material have formed, optionally and preferably at least one rotating portion or at least one filtering portion will hold and disassemble the occluding and thrombic material while preferably preventing any new material from reforming.

According to a still further preferred embodiment, preferably the rotating portion is coupled to the filtering portion to preferably prevent the formation of thrombi and occlusion material on the filtering portion. Optionally the blood clot filtering portion is deployed between the rotating portion blades and fixedly coupled to the rotating portion, and thereby preferably rotates together with the rotating portion. The filtering portion is preferably formed from a plurality of elongated strands arranged to form a generally filtering structure to capture and hold the blood clots flowing in the blood stream. According to a further optional embodiment, the filter portion may be formed as a net for filtering the particulate matter, for example by forming a basket-like conic structure as already widely used. For example, the filter portion may be manufactured from a flexible thread such as surgical monofilament sutures suitable for insertion into the body and/or for medical use. Other materials may optionally be used. For example and without limitation, metallic material, such as titanium, gold, and/or suitable alloys may optionally be used.

According to a still further embodiment of the present invention, optionally a filter portion may be fit to the device in various ways. For example, the rotating portion may optionally be adjacent to the filtering portion where optionally both the rotating portion and the filtering portion are not fixedly coupled, such that the filtering portions do not rotate with the rotating portion. The rotating portions may optionally be fitted adjacent to the filtering portion and placed at either side of the filter portion, and most preferably, when there is more than one rotating portion, the rotating portions may be placed on both sides of the filter portion.

In a further embodiment, the present invention is directed to the prevention of the occurrence, or the recurrence, of cerebral vascular diseases, particularly of stroke, comprising preventing the flow of embolic material flowing in the CCA from accessing the ICA, by performing one or more of routing and deflecting the flow of embolic material into the ECA, capturing and disassembling embolic material flowing towards the ICA and preventing the forming of occluding and thrombic material that may obstruct the blood flow or flow into the ICA.

More preferably, the device prevents the flow of embolic material flowing in the CCA from accessing the ICA and the ECA by capturing and disassembling embolic material flowing towards the CCA and preventing the forming of occluding and thrombic material that may obstruct the blood flow or flow into the ICA and ECA.

Prevention of cerebral vascular disease is preferably achieved by implanting, permanently or temporarily, in the vicinity of the bifurcation of the common carotid artery (CCA) into the internal carotid artery (ICA) and the external carotid artery (ECA) or the CCA or the ICA, a device according to some embodiments of the present invention.

It should be emphasized that while the described embodiments throughout this specification reference are made mostly to the carotid artery and the inferior vena cava, this is done for the sake of brevity only, but the invention is in no way limited to this specific location. The device of the invention can be advantageously be used at any other suitable blood vessels or bifurcation of blood vessels as existing, for instance, in the leg or the aortic arch.

Also it should be emphasized that although the described embodiments refer to blood vessels and blood flow, the present invention may also optionally be employed within other lumen(s) of the body, preferably those lumen(s) through which there is fluid flow, including but not limited to the cerebrospinal fluid system, the lymphatic system, the gastrointestinal tract, the kidneys and bladder (urinary tract and associated system), the male reproductive tract, fluid flow within the eyes and so forth.

A device or one of its components according to any one of the embodiments of the present invention may optionally be used either temporarily for acute conditions or procedures, or permanently for chronic conditions or long term treatment. For example, the device may optionally be used temporarily to protect against embolic material circulating in the blood steam during surgery. Preferably the device may be optionally retrieved after the procedure by medical instruments well known in the art, for example optionally by including an introducer catheter that is re-advanced over a guidewire to the site of the device in order to retrieve the device or alternatively to press its components within the vessel support structure against the vessel wall. The guidewire may optionally be retracted proximally such that the locking mechanism of the guidewire interacts with the vessel supporting structure or the rotating portion mechanism of the device and pulls the device proximally either partially or completely into the introducer catheter. The device is then removed from the vasculature.

Alternatively, for example, following the completion of the therapeutic or diagnostic procedure, an angioplasty catheter with distal introducer may optionally be used to retrieve the device by withdrawing the guidewire. Optionally and preferably the filter device is preferably retrievable. Optionally the filter portion may also be collapsed and removed from the blood vessel, preferably also removing any embolic debris trapped within any one of its components preferably the filter portion. A recovery sheath can be delivered over the guide wire using over-the-wire techniques to collapse the expanded filter for removal from the patient's vasculature as known in the art and incorporated herein by reference.

Alternatively, during short term implementation, the device in any one of its embodiments may be placed in the vasculature during a procedure while it is optionally kept in place by being attached to the distal end of an elongated member, for example including but not limited to a guidewire, once the procedure is concluded, optionally the device in any one of its embodiments of the present invention may be retrieved.

A further feature of the present invention in any one of its embodiments is its temporary use, wherein only the rotating portion and/or the filter portion are retrieved by medical instrument or intervention, for example including but not limited to an angioplasty catheter with distal introducer. The rotating portion and/or the filter portion are then placed back into the distal introducer in a manner similar to the other embodiments already described, preferably by using the locking mechanism of the guidewire to interact with the rotating portion and/or the filter portion, and then pulling the rotating portion and/or the filter portion proximally either partially or completely into the introducer catheter.

A further exemplary embodiment is by using a non-compliant balloon that can be inflated against the vessel walls in order to spread the luminal components such as the rotating portion against the vessel wall. Optionally the rotating portion may be securely attached and removed from an associated object, including but not limited to vessel support structure, filter portion, secondary object or the like with a mechanical assembly, including but not limited to a turn lock assembly or a pin lock assembly. More preferably such attachment and removal is performed by taking advantage of the device's material properties that can optionally be triggered in response to changes in heat, cold, pH, electric or magnetic fields in the vicinity of the rotating portion applied by external source. Optionally the rotating portion may be controllably released with any combination thereof.

Any of the embodiments of the present invention may also optionally be used as an adjunct during surgery, for example with the addition of a temporary device and/or temporary element of the device. This temporary device and/or temporary portion of the device may be retrieved after surgery. For example, a vessel support structure incorporated with a rotating portion according to the present invention may optionally be coupled to a passive filter, for example including but not limited to a conic mesh filter or the like and deployed during a procedure, therefore preferably providing double stage protection where the rotating portion is an active filter while the conic mesh filter acts as a passive filter.

Optionally the device vessel support structure is shaped as an airfoil on internal surfaces of the vessel support structure for increasing blood flow through the rotating portion and further deflecting embolic material into the rotating portion and filter portion.

Any of the embodiments of the present invention may optionally have various medical applications, including but not limited to, prevention or treatment of blockage and/or embolic material of any blood vessel or any other bodily passage, such as the carotid artery, aorta, veins and so forth; prevention or treatment of blockage and/or embolic material of any blood vessel or any other bodily passage which is secondary to medical treatment, such as catheterization; and use of the device to overcome medical conditions which may cause or exacerbate the formation of blood clots.

Optionally the device of the present invention in any of its embodiments comprising the vessel support structure and the rotating portion may preferably but optionally be composed of metallic material, for example including but not limited to 300 series stainless steels, platinum, platinum-iridium alloys, cobalt-chromium alloys such as MP35N, unalloyed titanium, or the like.

Optionally the device of the present invention in any one of its embodiments may optionally in whole or in part may be composed of plastic material for example including but not limited to a shape memory alloy (SMA) such as but not limited to nickel titanium alloy (NiTi) also known as nitinol, having a transition temperature around body temperature or a shape memory polymer (SMP) that can optionally be triggered in response to changes in heat, pH, electric or magnetic fields. Optionally any other materials or any combination thereof may be used.

Optionally the filter portion of the device of the present invention in any one of its embodiments optionally in whole or in part may be comprised from at least one layer. The filter layers may optionally not be joined but rather overlapping each other, thereby trapping embolic material flowing in the blood, while providing passage for medical instruments such as diagnostic and/or therapeutic catheters through the filter device and the filter portion.

The device of the present invention in any of its embodiments may optionally be coupled to various objects and therefore may serve as a platform for introducing secondary objects for example including but not limited to sensors, medicaments, small scale devices, or the like. Most preferably the secondary objects are coupled to the rotating portion of the device of the present invention preferably utilizing the rotational speed to control the secondary device. For example, the rotational speed of the rotating portion may optionally and preferably act as a trigger to release the medicament, electrical current, or to communicate sensed data for example blood pressure or the like.

Optionally the device of the present invention, in any of its embodiments, may be coupled to the secondary object by various means that are conducive both to the secondary object and the device itself, for example coupling according to any suitable method including but not limited to loading, or coating or the like.

For example, the device of the present invention, in any of its embodiments, may optionally serve as a platform for carrying at least one sensor for example including but not limited to physiologic sensor, hematological sensor, biochemical sensor and like sensors. Preferably and optionally a sensor may be coupled to the rotating portion of the device of the present invention. The sensor will optionally enable continued monitoring of at least one parameter for example including but not limited to temperature, blood pressure, heart rhythm, pH, electrolytes, blood sugar, blood cholesterol levels or the like.

The device of the present invention in any of its embodiments may optionally be coupled with a medicament for example including but not limited to blood thinning drugs, hormones, genes or the like. Most preferably the medicament is coupled to the rotating portion of the device. Optionally the device may be loaded with a medicament optionally by coating or by coupling small aggregates that release the medicament for example including but not limited to hormones, genes or the like. Optionally the release of the medicament may preferably be controlled by the activity of the rotating portion of the device and optionally include automatic release, uniform release, or any type of reaction for example a magnetic reaction, rotating speed reaction (high or low blood flow), servomechanism, sensor programming or external control, optionally through a dispenser and/or medicament release mechanism for example.

In any some of the embodiments of the present invention, the device may act as a foreign body that may stimulate the formation of thrombi and occluding material. Similarly, the formation of thrombi may be instigated due to large embolus caught in the filter portion of the device. Accordingly, the deployment of the device of the present invention may optionally be coupled to a course of drug treatment, for example including but not limited to drugs that inhibit or control the formation of thrombus or thrombolytics such as heparin or heparin fragments, aspirin, coumadin, tissue plasminogen activator (TPA), urokinase, hirudin, and streptokinase, and other suitable therapies may be used.

The device of the present invention in any one of its embodiments may be optionally coated with a substance or structure that improves biocompatibility or tissue adaptation or coated for in vivo compatibility as is described and well known in the art. The device may be coated with one or more of the following: antiproliferatives (methotrexate, cisplatin, fluorouracil, Adriamycin, antioxidants (ascorbic acid, carotene, B, vitamin E, and the like), antimetabolites, thromboxane inhibitors, non-steroidal and steroidal anti-inflammatory drugs, Beta and Calcium channel blockers, genetic materials including DNA and RNA fragments, and complete expression genes, carbohydrates, and proteins including but not limited to antibodies (monoclonal or polyclonal) lymphokines, growth factors, prostaglandins, and leukotrienes, and other suitable therapies may be used.

The device of the present invention in any one of its embodiments may optionally in whole or in part be formulated or composed of biodegradable material, preferably reducing the risk of thrombus formation and reducing need for chronic implantation of vessel support structures. Optionally the biodegradable material may be composed of materials such as polylactic acid polyglycolic acid (PGA), collagen or other connective proteins or natural materials, magnesium alloys, polycaprolactone, hylauric acid, adhesive proteins, co-polymers of these materials as well as composites and combinations thereof and combinations of other biodegradable polymers, biodegradable glass, bioactive glass or the like. Preferably, biodegradable glass, bioactive glass is also a suitable biodegradable material for use in the present invention.

For example, the vessel support portion of the device may optionally be composed of biodegradable material wherein preferably over time the support structure is absorbed by the body after healing of the angioplasty site. Absorption into the vessel wall is preferred as it avoids the limitation of current vessel support structures, such as a stent, by alleviating the need for chronic implantation. Optionally and preferably it would be further desirable to use biodegradable material that could optionally be shaped in a desirable manner, for example including but not limited to a mesh-like or porous configuration, that would optionally enable endothelial cells at the angioplasty site to grow into and over the vessel supporting structure so that bio-degradation will occur within the vessel wall. Similarly, any portion of the device may be formed with biodegradable matter for example including but not limited to the rotation portion, that may optionally be degraded over time or optionally by a triggering event such that the biodegradation will only start upon a cue for example when the site is deemed to be healed. For example, in some implementations such as stent or stent-graft or an in-stent restenosis (ISA), the rotating portion of the device may optionally not be required beyond a certain period of time. Optionally in such implementation the rotating portion may be composed of biodegradable material to minimize the risk of thrombus formation and other complications.

According to optional embodiments of the present invention, there is provided a method of treating occluding matter within a blood vessel, comprising implanting a device comprising a movable blade within the blood vessel; permitting blood to flow through the movable blade; and moving the movable blade by the blood flow, wherein a movement of the movable blade breaks up the occluding matter.

According to optional embodiments of the present invention, there is provided a method for determining at least one cardiac parameter, comprising: implanting a device comprising a movable blade within the blood vessel; permitting blood to flow through the movable blade; moving the movable blade by the blood flow; and determining the at least one cardiac parameter according to moving of the movable blade.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A-C are schematic diagrams of an exemplary miniature rotating portion according to an optional embodiment of the present invention; and

FIG. 2A-C are schematic diagrams of an exemplary miniature rotating portion according to an optional embodiment of the present invention; and

FIG. 3A-C are schematic diagrams of an exemplary miniature rotating portion according to an optional preferred embodiment of the present invention; and

FIG. 4 is a schematic diagram of an exemplary mounting orientation of the miniature rotating portion according to an optional embodiment of the present invention; and

FIG. 5A-F are exemplary schematic diagrams of the blades used in the rotating portion according to an exemplary embodiment of the present invention; and

FIG. 6A-G is an exemplary schematic diagram of the vessel supporting structure according to an exemplary embodiment of the present invention as implanted in a blood vessel; and

FIG. 7A-C and 8A-C depict an alternative optional mode of disabling the rotation portion of the preferred embodiment of the present invention; and

FIG. 9 depicts an optional mode of device delivery and realigning of the preferred embodiment of the present invention; and

FIG. 10A-B are exemplary schematic diagrams of the rotating portion of the device according to an exemplary embodiment of the present invention; and

FIG. 11A-B are exemplary schematic diagrams of an implanted filter device according to an exemplary embodiment of the present invention; and

FIG. 12A-D are exemplary schematic diagrams of an implanted filter device in a bifurcated vessel according to an optional embodiment of the present invention; and

FIG. 13 is exemplary schematic diagrams of an implanted IVC filter comprising of the rotating portion being used with as an active filter where the device vessel support structure acts also as a passive filtering device; and

FIG. 14A-C are exemplary schematic diagrams of exemplary embodiments of the present invention based on rotating portion placement.

FIG. 15A-C are exemplary schematic diagrams of different views of placement of a plurality of rotating portions according to an exemplary embodiment of the present invention; and

FIG. 16A-C are exemplary schematic diagrams of an implanted filter device of different views of an exemplary embodiments of the present invention; and

FIG. 17A-C are exemplary schematic diagrams of an implanted filter device of different vessel support structure and filter portion of an exemplary embodiments of the present invention; and

FIG. 18A-E are exemplary schematic diagrams of vessel support structure ring and a vessel support structure ring incorporated with a filter portion according to exemplary embodiments of the present invention; and

FIG. 19A-D are exemplary schematic diagrams of the delivery method according of an exemplary embodiment of the present invention; and

FIG. 20A-D are exemplary schematic diagrams of the delivery method according of an exemplary embodiment of the present invention;

FIG. 21A-D are exemplary schematic diagrams of the delivery method according of an exemplary embodiment of the present invention; and

FIG. 22 relates to an exemplary schematic diagram of a device according to an embodiment of the present invention in which transverse rotation occurs.

### DESCRIPTION OF THE PREFERRED EMBODIMENTS

According to preferred embodiments, the present invention is of a system and a method for a hydrokinetic rotating device that is implanted in a blood vessel within a vessel support structure, including but not limited to a stent, blood clot filter, wire frame and stent-graft. The rotating portion may optionally be coupled to a number of devices having variable uses, including but not limited to a novel stent device which prevents restenosis and an active embolic protection device or to a novel blood clot protection device which effectively captures blood clots and promotes blood clot lysis.

The principles of the present invention may be better understood with reference to the drawings and the accompanying description.

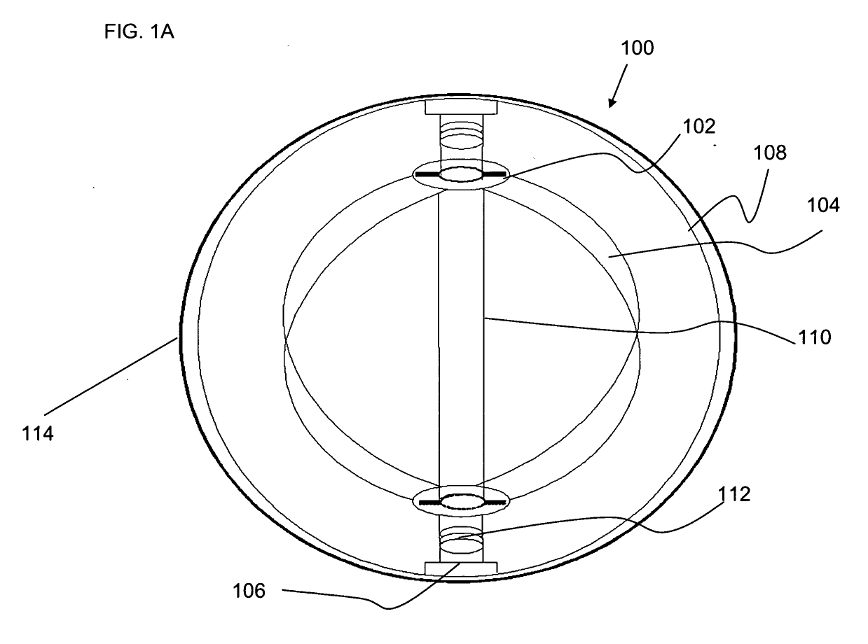

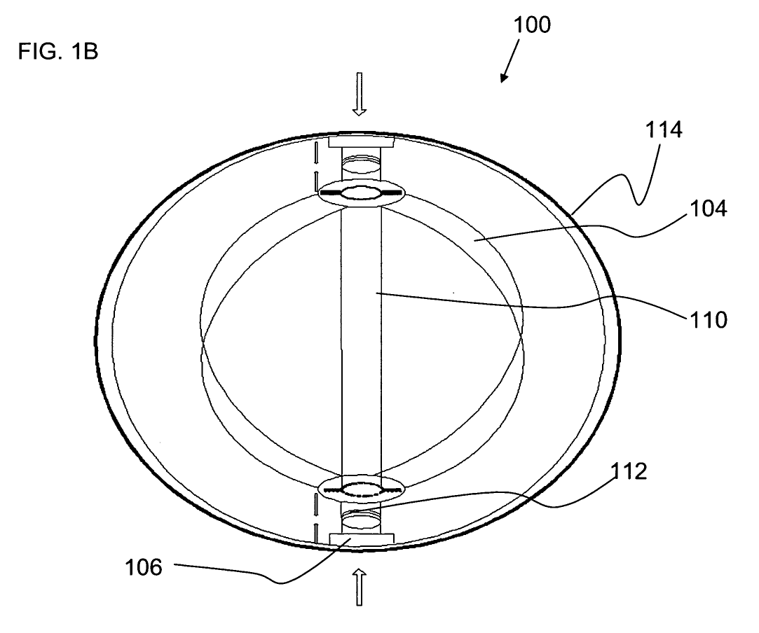

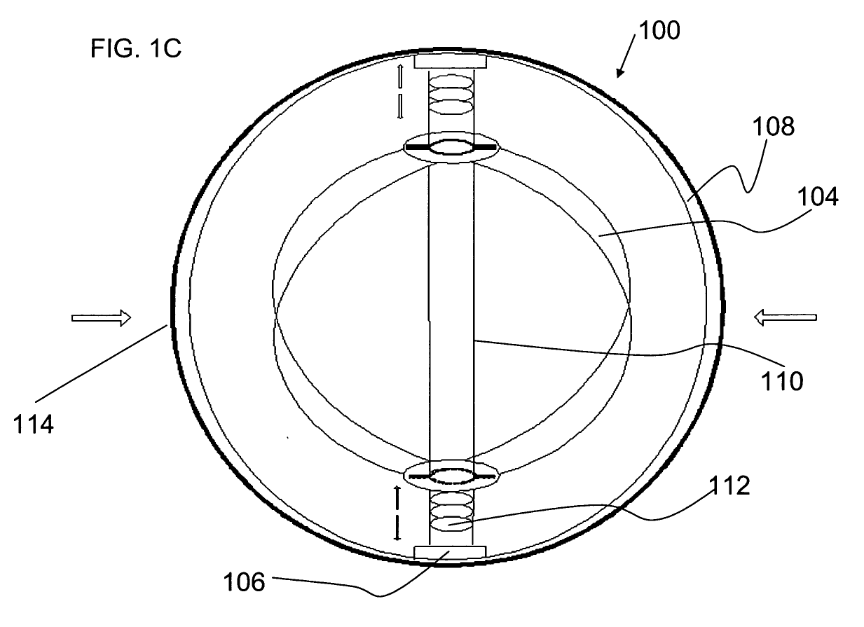

Referring now to the drawings, FIG. 1A is a schematic cross section diagram of an exemplary rotating portion 100 according to an optional embodiment of the present invention. Rotating portion 100 is attached to a vessel support structure 108, preferably including but not limited to a tubular structure, a stent, blood clot filter, wire frame and stent-graft; vessel support structure is provided as a non-limiting example of a support structure. Vessel support structure 108 is inserted within the lumen of a blood vessel wall 114, preferably according to the stent introducing techniques known in the art, and incorporated herein by reference, utilizing a guiding catheter, a guide wire and a balloon angioplasty catheter. The stent delivery system that includes the stent is optionally and preferably advanced over the guide wire and the stent is then deployed at the site of the dilated stenosis (not shown).

Once in place within the blood vessel walls 114, the rotating portion 100 keeps the lumen open allowing blood to flow therethrough. Furthermore the movement of blades 104 preferably captures and disassembles any plaque or occluding material flowing through or building up in the lumen of vessel support structure 108. A rotating portion axis 110 preferably spans the diameter of the vessel walls 114, and is preferably positioned transversely and perpendicularly to the direction of blood flow to produce rotation in a plane parallel to the direction of blood flow. Rotating portion 100 is preferably anchored to vessel support structure 108 by at least one anchor 106. Blades 104 are preferably coupled to rotating portion axis 110 through rotors 102 which are optionally molded or otherwise integrally formed with blade 104 to create a uniform structure. Blades 104 are optionally shaped to maximize rotational speed and/or to provide a specific energy generation efficiency. Preferably blades 104 are helical and airfoil-shaped, and spherically or elliptically warped. As the blood flows through the lumen of the vessel support structure 108, it causes blades 104 and rotors 102 to spin. It is the spinning blades 104 and rotors 102 that define the operation of rotating portion 100 with respect to maintaining the lumen of vessel 114 free of potential blockages. As potential occluding material passes through the vessel support structure 108, rotating portion 100 disassembles and breaks it up into smaller pieces, for example allowing the body to digest or absorb it naturally (or otherwise allowing it to be removed from the blood stream). Furthermore, anchor 106 increases overall stability of vessel support structure 108 and vessel walls 114.

However, because vessel walls 114 are not static and immobile, rotating portion 100 and vessel support structure 108 are preferably sufficiently flexible to accommodate for the natural forces acting on vessel walls 114. Accordingly, at least one flexible support structure 112, which may also optionally be described as a shaft, optionally including but not limited to a spring like element, is integrated between axis 110 and anchor 106, that provide rotating portion 100 with the required flexibility, allowing it to size itself according to the changing shape of vessel walls 114 while providing support structure 108 with sufficient structural integrity.

An optional but preferred extent of flexibility of rotating portion 100 is shown in FIGS. 1b-c. Accordingly, rotating portion structure 100 is preferably able to adjust one or more dimensions in accordance with the size of blood vessel walls 114. Flexible support structure 112 and flexible blades 104 preferably provide the structure of rotating portion 100 with the desired flexibility, allowing the rotating portion 100 to reshape itself in accordance with the various forces acting on the vessel walls 114.

FIG. 1B is an exemplary depiction of how rotating portion 100 and vessel support structure 108 may optionally and preferably be reshaped (i.e. to have a change in at least one dimension) in accordance with vertical forces that act on the vessel walls 114. Flexible structure 112 and flexible blades 104 absorb the applied vertical force, and preferably adjust at least one dimension to condense and hence to reshape rotating portion 100 to appropriately fit vessel walls 114.

Similarly, FIG. 1C is an exemplary depiction of how rotating portion 100 may optionally and preferably be reshaped (i.e. to have a change in at least one dimension) in response to horizontal forces acting on vessel walls 114. Flexible support structure 112 and flexible blades 104 preferably absorb the applied vertical force and expand, thereby adjusting at least one dimension to reshape rotating portion 100 to better fit vessel walls 114. Thus, blades 104, optionally made of pliable material, also preferably change configuration to better fit vessel walls 114 as constriction forces are applied on blades 104 both vertically and horizontally. Rotating portion 100 is optionally made of pliable material, allowing rotating portion 100 to be reshaped numerous times.

Blades 104 are optionally and preferably made of pliable material, optionally including but not limited to magnetic material incorporated within the blades 104, and/or a magnetic coating on the outer surface of blade 104 which may optionally be used to generate electric charge. Optionally, blades 104 and rotors 102 may generate an electric charge in order to further prevent blood components from collecting, optionally by incorporating or integrating metals and/or polymers that are naturally charged, or by incorporating piezo-electric materials which may generate electric potential that generate electric charge by the rotating action.

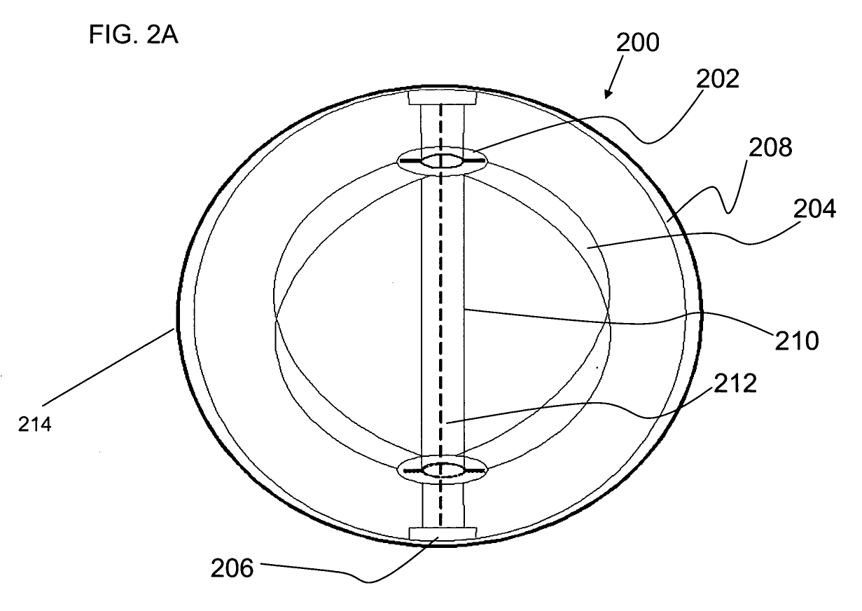

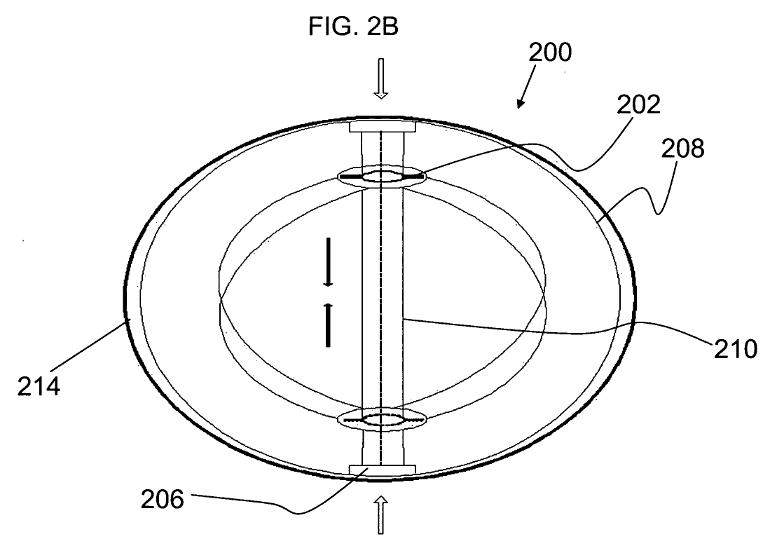

An additional embodiment of rotating portion 100 of FIG. 1 is shown in FIG. 2 that shows an optional, exemplary embodiment of the present invention. A rotating portion 200 is implanted within the lumen of a blood vessel wall 214, and as described above that is supported by a vessel support structure 208, preferably including but not limited to a tubular structure, a stent, blood clot filter, wire frame and stent-graft. A rotating portion axis 210 is preferably made of pliable or flexible material, and more preferably spans the diameter of the vessel support structure 208 lumen. Rotating portion axis 210 is also preferably positioned transversely to the direction of fluid flow, producing rotation in a plane parallel to the direction of fluid flow. Rotating portion axis 210 is optionally and preferably held in position relative to vessel support structure 208 by at least one anchor 206. Along rotating portion axis 210 at least one and more preferably two rotors 202 are optionally molded with and/or integrally formed with at least one blade 204, that is optionally an airfoil-shaped spherically warped blade. As blood flows through the lumen of vessel support structure 208, it applies rotational forces on blades 204, causing them to spin and in turn spinning rotors 202. Axis 210 is preferably made from pliable material and provides rotating portion 200 with flexibility, allowing it to size itself (i.e. to change at least one dimension) according to the changing shape of vessel walls 214, as depicted in FIGS. 2B-C and also as noted previously with regard to FIG. 1.

Vessel walls 214 are not static, such that their shape is modified as constrictive forces are applied on them. Accordingly, rotating portion structure 200 is preferably able to resize itself (i.e. to change at least one dimension) in accordance with the size of blood vessel walls 214. Axis 210 preferably provides rotating portion structure 200 with the required flexibility and structure for such alteration of at least one dimension.

FIG. 2B depicts how the rotating portion may optionally and preferably reshape (i.e. to change at least one dimension) in accordance with vertical forces that act on the vessel walls 214. Axis 210 preferably absorbs the applied vertical force and condenses to reshape device 200 to fit vessel 214.

Similarly, FIG. 2C is an exemplary depiction of how rotating portion 200 may optionally and preferably reshape (i.e. to change at least one dimension) in response to horizontal forces acting on vessel walls 214. Axis 210 preferably absorbs the applied vertical force and expands to reshape rotating portion 200 to fit vessel walls 214 reconfigured shape. Furthermore, blades 204, optionally molded and/or integrally formed with rotors 202, are optionally made of pliable material and able to change configuration to fit the new shape with the vessel walls 214 as constriction forces apply to it both vertically and horizontally. Preferably rotating portion 200 is optionally made of malleable and pliable material allowing it to be continuously reshaped.

Blades 204 are optionally made of pliable material, including but not limited to a magnetic material incorporated within the blades and/or magnetic coating on the outside of blades 204, that optionally functions to generate electric charge. Blades 204 and rotors 202 may optionally generate an electric charge in order to further prevent blood components from collecting optionally incorporating metals and/or polymers that are naturally charged, or by incorporating piezo-electric materials which may generate electric potential that generate electric charge by the rotating action.

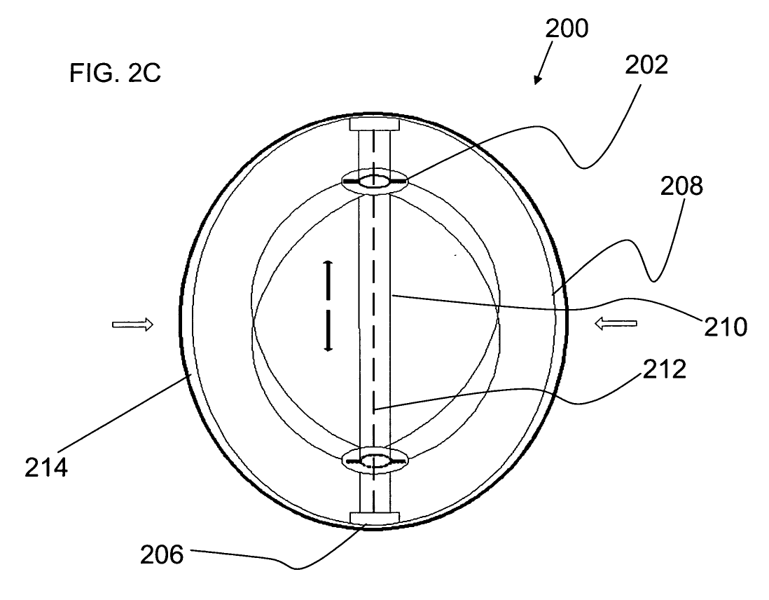

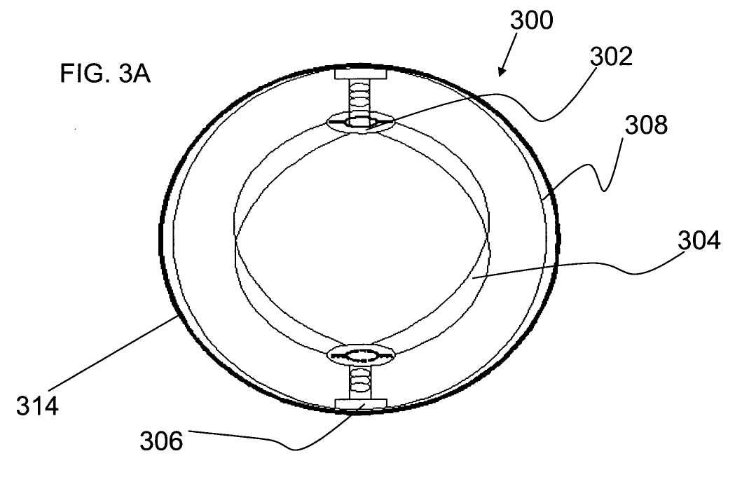

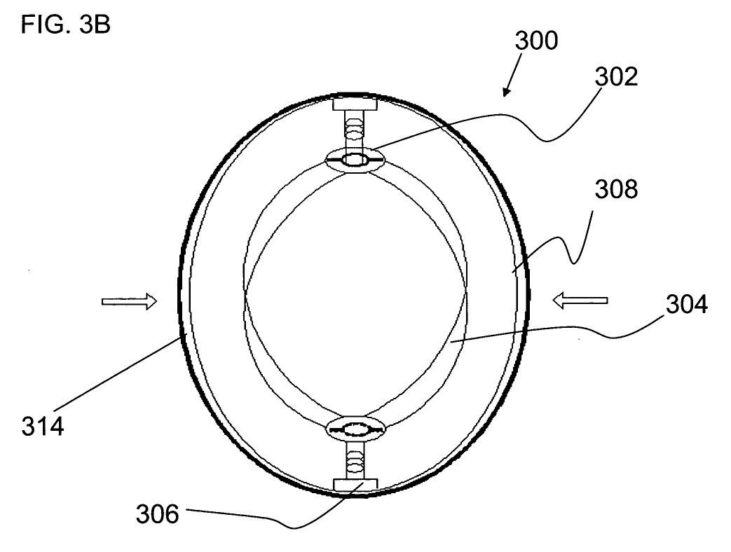

FIG. 3A presents a still further optional embodiment of the present invention that is another optional configuration of the rotating portion structure introduced in FIGS. 1 and 2 above. A rotating portion 300 is preferably implanted within the lumen of a blood vessel wall 314 supported by a vessel supporting structure 308, optionally including but not limited to a tubular structure, a stent, a coated stent, blood clot filter, wire frame and stent-graft. At least one support anchor 306 is preferably positioned transversely to the direction of fluid flow to produce rotation in a plane parallel to the direction of blood flow, while securing rotating portion 300 in position relative to vessel support structure 308. At least one and preferably two rotors 302 are positioned and optionally molded and/or integrally formed with at least one blade 304, which is optionally and preferably helical and airfoil-shaped, and is more preferably spherically warped. As blood flows through the lumen of vessel support structure 308, it applies a force on blades 304 causing them to spin, and in turn this causes rotors 302 to spin. Blade 304, preferably made of pliable material, provides rotating portion 300 with flexibility allowing it to size itself (i.e. to change at least one dimension) according to the changing shape of vessel walls 314.

FIG. 3B is an exemplary depiction of how rotating portion 300 may reshape in response to vertical forces that act on the vessel walls 314. Anchors 306 preferably absorb the applied vertical force and condense (i.e. to change at least one dimension) to reshape rotating portion 300 to fit vessel 314.

Similarly, FIG. 3C exemplary depicts how rotating portion 300 may optionally and preferably reshape in accordance with horizontal forces acting on vessel walls 314. Anchors 306 preferably absorb the applied vertical force and expand to reshape (i.e. to change at least one dimension of) rotating portion 300 to fit a reconfigured shape of vessel walls 314. Furthermore, blades 304 preferably change configuration to fit the new shape with the vessel walls 314 as constriction forces act on blades 304 both vertically and horizontally. Preferably rotating portion 300 is optionally made of malleable and pliable material allowing it to be repeatedly reshaped.

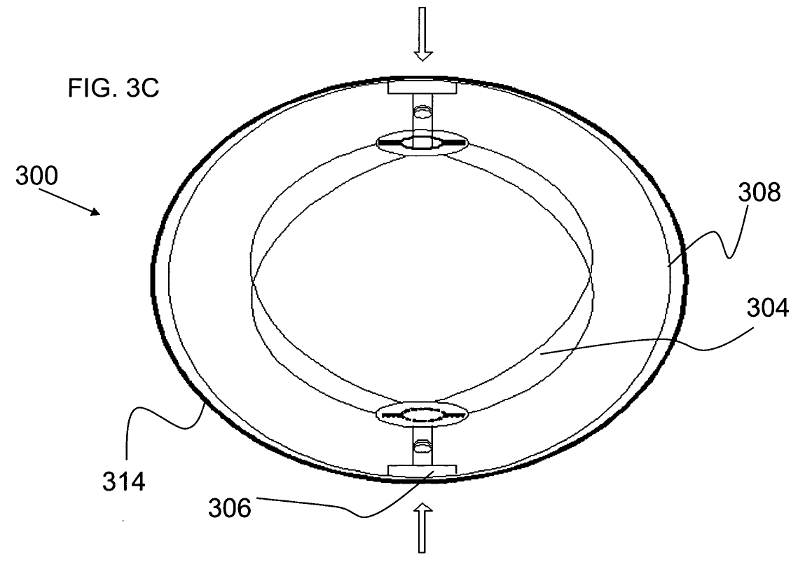

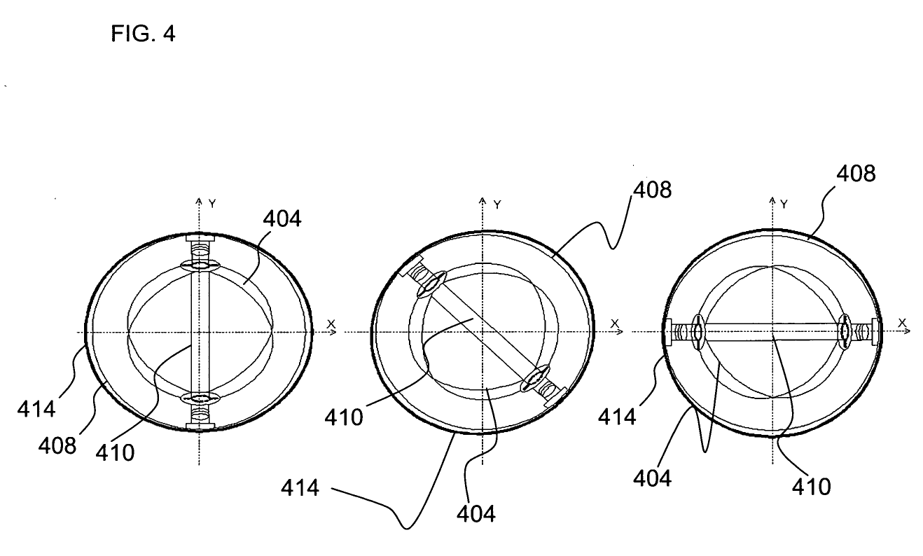

FIG. 4 shows an optional non-limiting embodiment of rotating portion, with regard to mounting of the embodiment described in FIG. 1 above, such that rotating portion axis 110 (of FIG. 1) has been rotated 90 degrees to produce rotating portion axis 410 having a horizontal orientation. This rotation shows that a rotating portion according to any one of the embodiments of the present invention may be oriented in any manner within the vessel supporting structure 408 and blood vessel walls 414 as the shape of blade 404 determines the rotational direction.

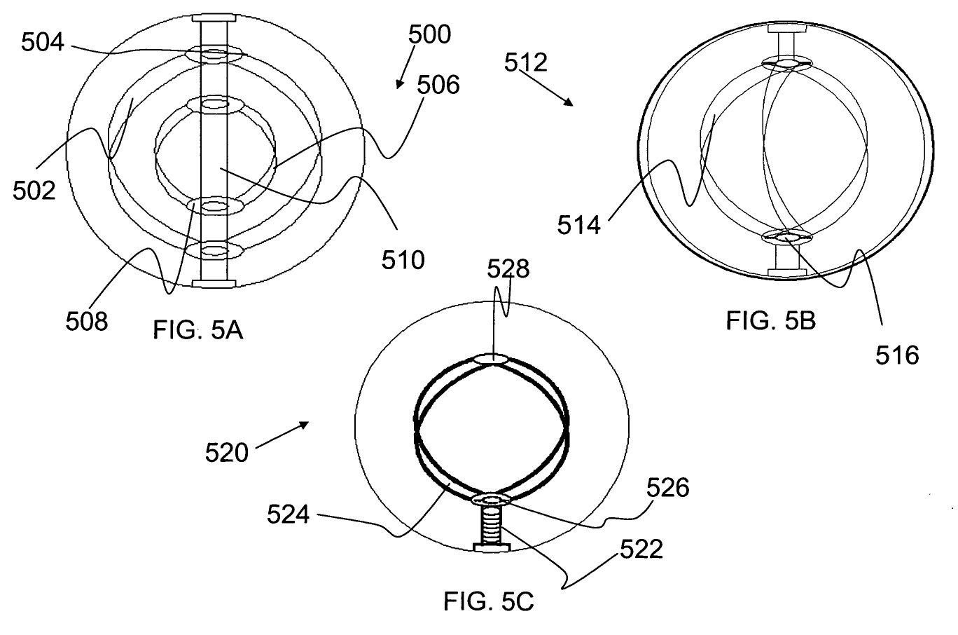

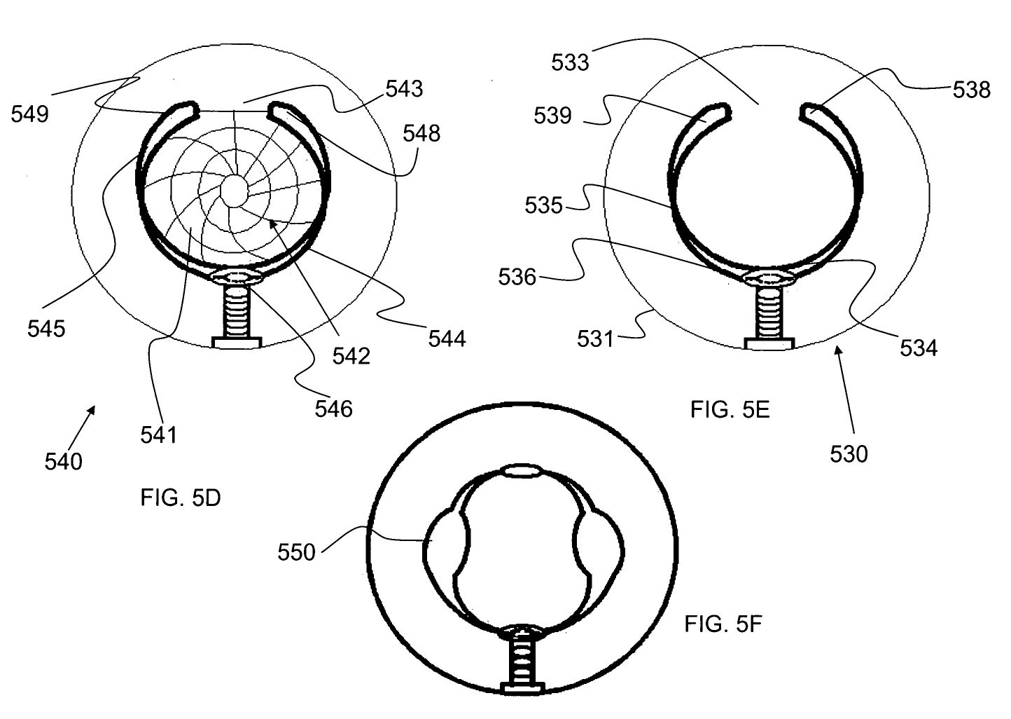

FIG. 5A is a depiction of a still further non limiting embodiment of the blades of the present invention, having an exemplary multiple layer configuration 500. Multiple layer configuration 500 preferably comprises at least two concentric blades as shown, an inner blade 506 attached to axis 510 via rotors 508 (optionally molded and/or integrally formed with inner blade 506), and an outer blade 502 optionally molded and/or integrally formed with rotors 504. The adjacent blades, inner blades 502 and outer blades 506, are preferably shifted circumferentially such that they do not overlap each other during rotation. That is, inner blades 506 preferably generate a spherical shaped rotating portion, which is positioned inside the outer spherical shaped rotating portion. The radius of inner blades 506 is preferably always smaller than the radius of the outer blades 502. The multilayer arrangement increases the torque of rotating portion 500.

FIG. 5B is a depiction of a still further non limiting embodiment of the rotating portion blades having a triple blade configuration 512. Triple blade configuration 512 preferably has three blades 514 that are optionally molded with rotor 516. The triple blade configuration may optionally be implemented with any rotating portion assembly configuration of a non-limiting embodiment of the present invention. Other multiple blade configurations having a plurality of blade groupings may optionally and preferably be implemented within the present invention.

FIG. 5C is a depiction of a still further non limiting embodiment of the rotating portion blades having one anchor configuration 520. Single anchor configuration 520 comprises one flexible anchor 522 and one rotor 526 that is optionally molded and/or integrally formed with at least one blade 524. Optionally, blade 524 is fitted with a flexible attachment 528 allowing a plurality of blades to be connected thereto. Anchor configuration 520 may optionally be implemented with any of the rotating portion assembly configurations previously presented according to the non-limiting embodiments of the present invention.

FIG. 5D is a depiction of a still further optional non limiting embodiment of the rotating portion blades having one anchor and an open configuration with a filter assembly 540. Filter assembly 540 preferably features a rotor 546 which is positioned and optionally molded and/or integrally formed with at least one blade 544. The blade upper tip 548 is open and is preferably rounded to prevent damaging the vessel walls. There is preferably a safety gap 543 from upper tip 549 of the other blade 545, which is preferably present to prevent surgical equipment, including but not limited to a guide wire, balloon angioplasty catheter and stent delivery system, from being caught between the upper tips 548 and 549 of the rotating portion blades 544 and 545, respectively. The blood clot filtering portion 542 is preferably deployed between the rotating portion blades 544 and is more preferably coupled thereto.

Filtering portion 542 is optionally formed from a plurality of elongated strands 541, optionally arranged to form a net or web-like structure in order to catch and hold blood clot(s) and/or other material or debris that are flowing in the blood stream. Elongated strands 541 are optionally and preferably fixedly attached to one another only at the apex of the filtering portion 542. As known in the art, the elongated strands 541 may optionally be formed from metallic material such as titanium and nitinol (nickel-titanium alloy), plastically deformable material, temperature-sensitive shape memory material with a transition temperature around body temperature, flexible thread such as surgical monofilament sutures or any elastic material preferably having a core formed from radiopaque material suitable for insertion into the body. Filter assembly 540 may optionally be implemented with any rotating portion assembly configuration of a non limiting embodiment of the present invention.

FIG. 5E is a depiction of a still further optional and non limiting embodiment of the rotating portion blades having a single anchor open configuration 530. Single anchor open configuration 530 preferably comprises one rotor 536 optionally molded and/or integrally formed with at least one blade 534. Upper tip 538 of blade 534 is open and preferably rounded to prevent damage to vessel wall 531, more preferably having a safety gap 533 from the upper tip 539 of another blade 535, for example to prevent a balloon angioplasty catheter from being caught between upper tips 538 and 539 of the rotating portion blades 534 and 535. The one anchor open configuration 530 may optionally be implemented with any rotating portion assembly configuration of a non-limiting embodiment of the present invention.

FIG. 5F is a depiction of a still further optional non limiting embodiment of the rotating portion's blades 550 having variable width along its length. Preferably the blades may be designed to produce the required rotational speed and momentum.

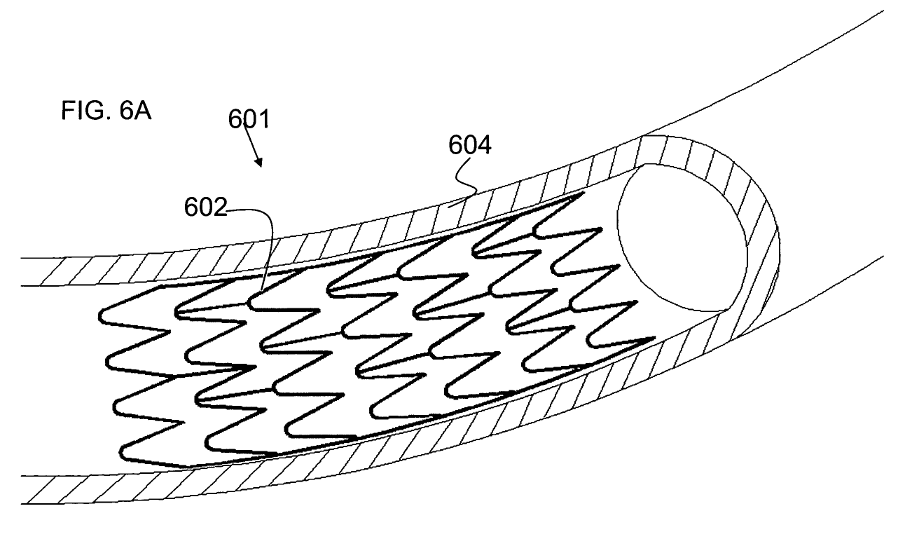

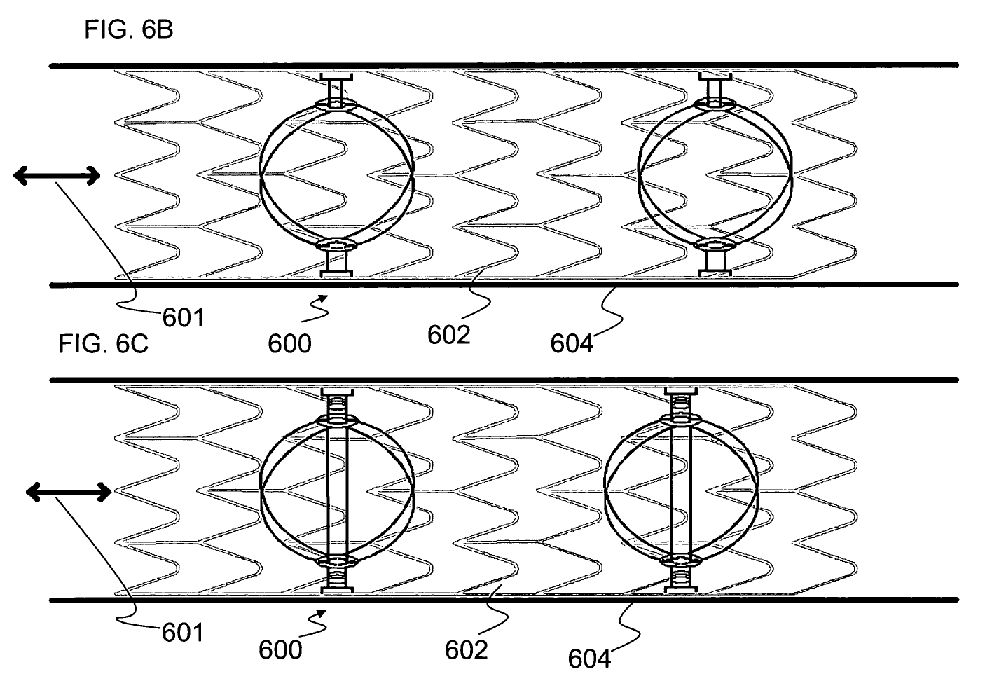

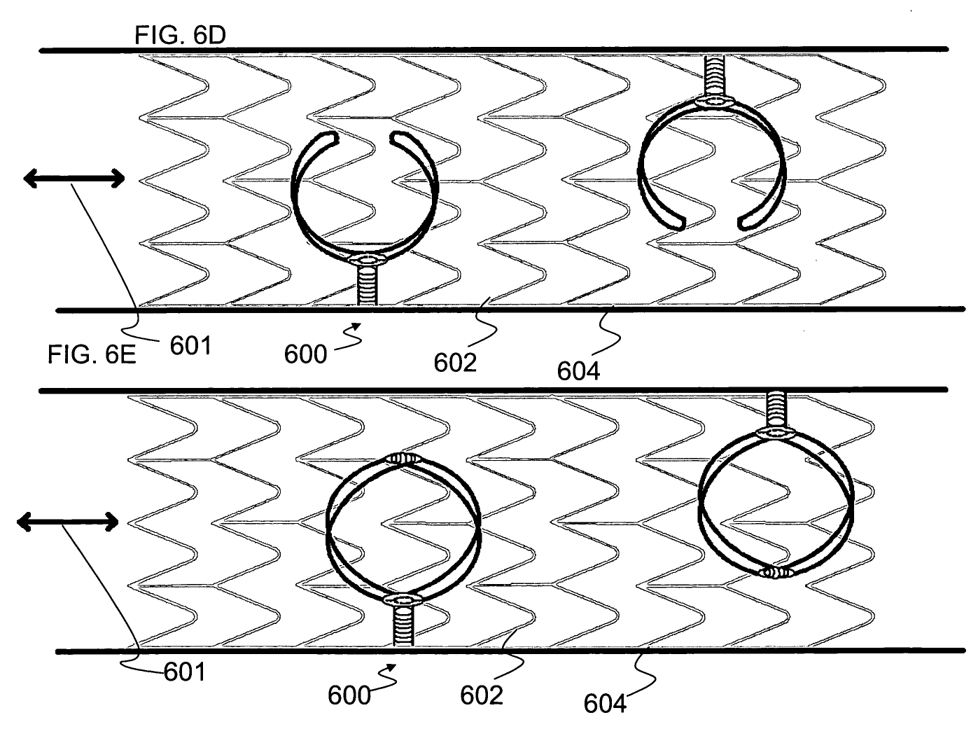

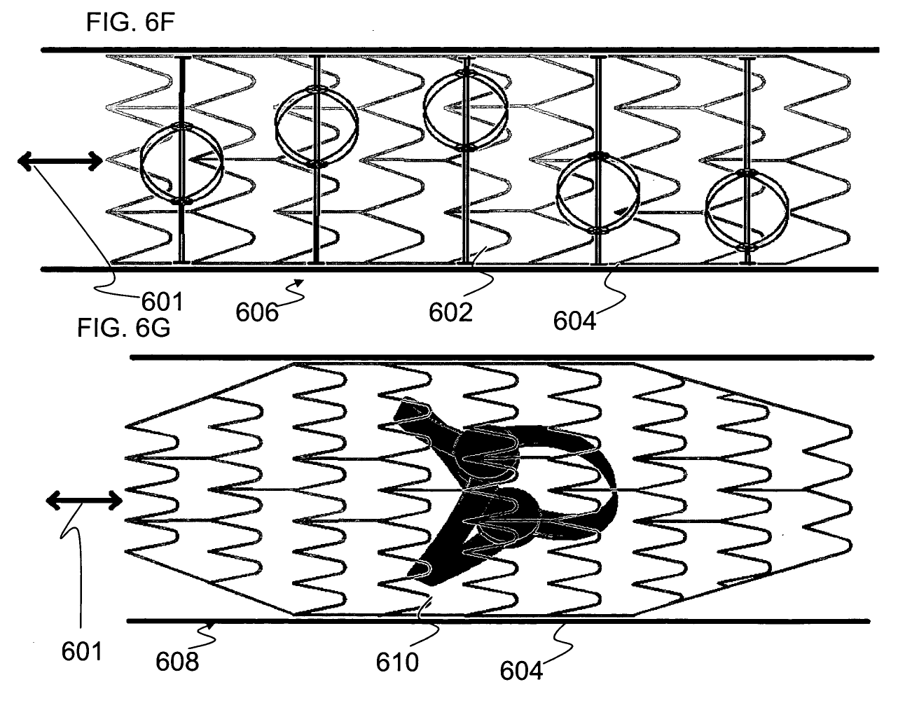

FIG. 6A is a cross-sectional view of a vessel supporting structure 602, which may optionally be implemented in any suitable form, preferably including but not limited to a tubular structure, a stent, blood clot filter, wire frame or stent-graft, imbedded in a blood vessel 601 having blood vessel walls 604. The vessel supporting structure 602 may optionally be fixed with a rotating portion structure (not shown). A rotating portion (not shown) according to the present invention preferably maintains both the structural integrity of blood vessel 601 while maintaining an open lumen allowing blood to flow freely through vessel support structure 602 as the rotating portion breaks down any plaques or other material that may form within or passing through vessel supporting structure 602.

FIG. 6B-G show an optional and exemplary implementation of the present invention, preferably comprising at least one rotating portion 600 according to any one of the rotating portion optional embodiments of the present invention as described in FIGS. 1-5 or any combination thereof in any orientation within a vessel supporting structure 602, which may optionally be implemented in any suitable form, including but not limited to tubular structure, a stent, blood clot filter, wire frame and stent-graft. Installation is within blood vessel wall 604 is preferably achieved by using a stent deployment method as is known in the art and incorporated herein by reference. Blood flows through vessel supporting structure 602 in blood flow direction 601, preferably causing the consecutive rotating portions 600 to spin, optionally and preferably preventing plaque from forming within the lumen of vessel 600, vessel walls 604 and/or vessel support structure 602.

According to a preferred non limiting embodiment of the present invention, a number of rotating portions 600 may be fitted into vessel supporting structure 602 each individually harnessing the hydrokinetic energy of blood flow in order to perform at least one of the rotating portions 600 various uses such as but not limited to maintaining an open lumen, filtering blood clots, generating electric charge and so forth.

The number of rotating portions 600 and their orientation in the vessel supporting structure 602 may optionally be varied to meet the various medical application requirements and to enable free blood flow through the vessel supporting structure 602. For example, in order to harvest hydrokinetic energy uniformly and preserve the laminar flow of blood, the rotating portions 600 may optionally be oriented in such way that each rotating portion 600 relates to a different portion of the blood vessel cross section (internal volume). Thus, by preferably not allowing the same blood “packages” or volumes to pass through more than one rotating portion 600, the hydrokinetic energy is harvested more uniformly.

Each rotating portion 600 may optionally be interconnected or function independently of the other. A greater number of rotating portions 600 housed within vessel support structure 602 is preferred as it provides a greater structural integrity.

FIG. 6F shows a system 606 featuring one or more rotating portions 600 that are able not only to spin but also to move vertically along and/or within vessel supporting structure 602.

FIG. 6G shows a system 608 featuring one or more rotating portions 600 that spin freely within the vessel support structure 610 and are enclosed by the vessel support structure 610.

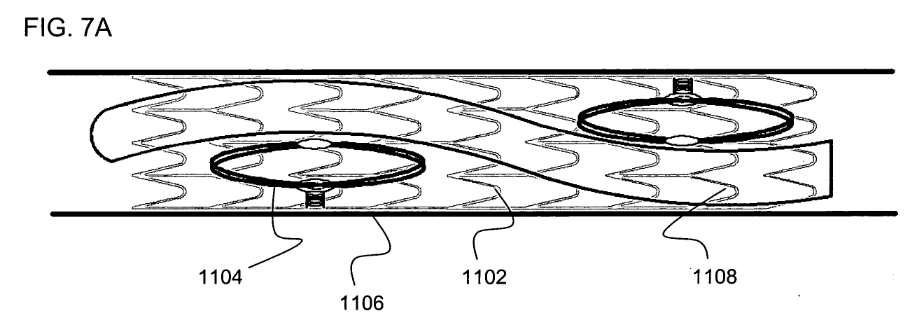

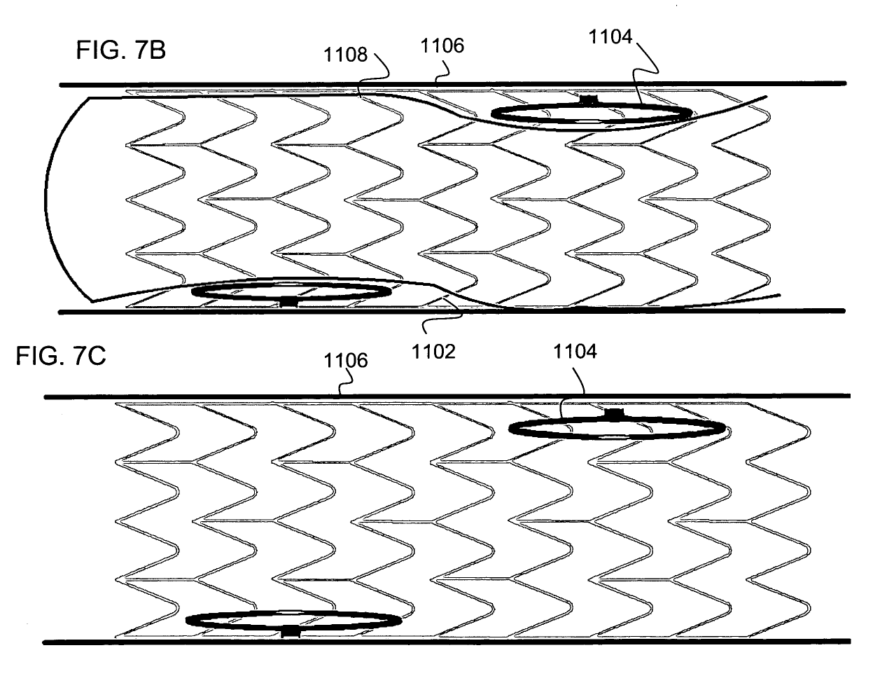

FIG. 7 depicts an optional mode of disabling and condensing the rotating portion of the preferred embodiment of the present invention. Optionally rotating portion 1104 is disabled and condensed by using for example a non-compliant balloon 1108 that may be inflated against the vessel walls 1106 to spread rotating portion 1104 or any other luminal components against the vessel walls 1106. Optionally, the condensed rotating portion and/or filter portion may then be removed from the vasculature. FIG. 7A depicts the non-compliant balloon 1108 entry into the lumen of vessel support structure 1102. FIG. 7B depicts the inflation of the non-compliant balloon 1108 that expands rotating portion 1104 within vessel support structure 1102 spreading it against vessel walls 1106, effectively disabling and expanding it while opening the lumen of vessel structure 1102. FIG. 7C depicts rotating portion 1104 in its expanded state against the vessel wall 1106, after balloon 1108 has been removed.

FIG. 8 depicts another example of how a different form of rotation portion 1104 according to the present invention may be disabled and spread within vessel support structure 1102 by using non compliant balloon 1108. Rotating portion 1104 is preferably disabled by inflating the non compliant lo balloon 1108, preferably by radial inflation against vessel support structure 1102. FIG. 8A depicts the non-compliant balloon 1108 entry into the lumen of vessel support structure 1102 threaded through rotating portion 1104. FIG. 8B depicts the inflation of balloon 1108 producing radial pressure that spreads rotating portion 1104 against the walls of vessel support structure 1102, effectively disabling and spreading it while opening the lumen of vessel structure 1102. FIG. 8C depicts rotating portion 1104 in its expanded state against vessel wall 1106, after balloon 1108 has been removed.

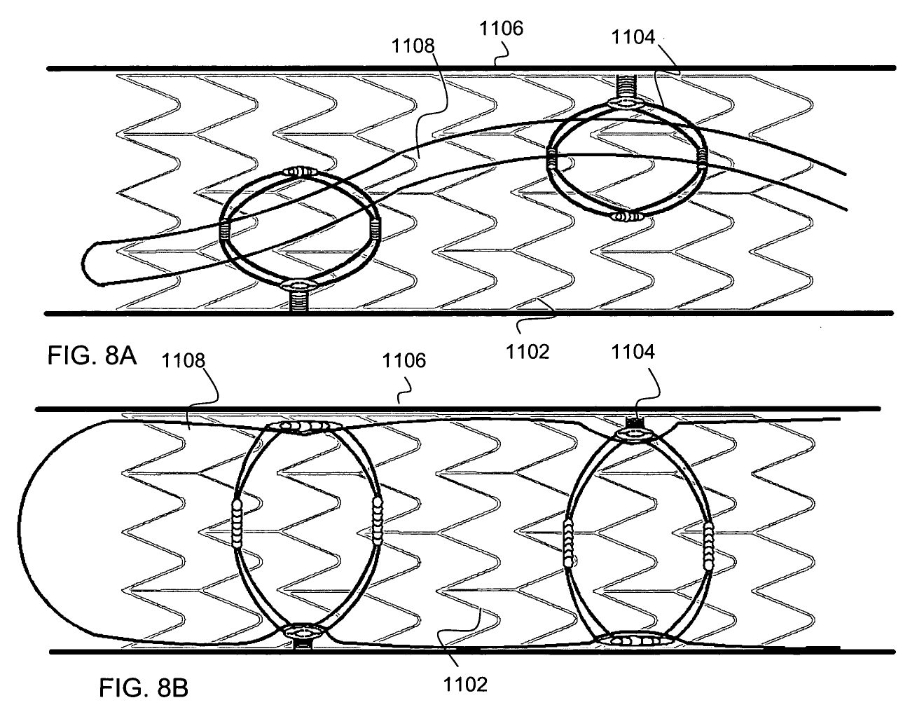

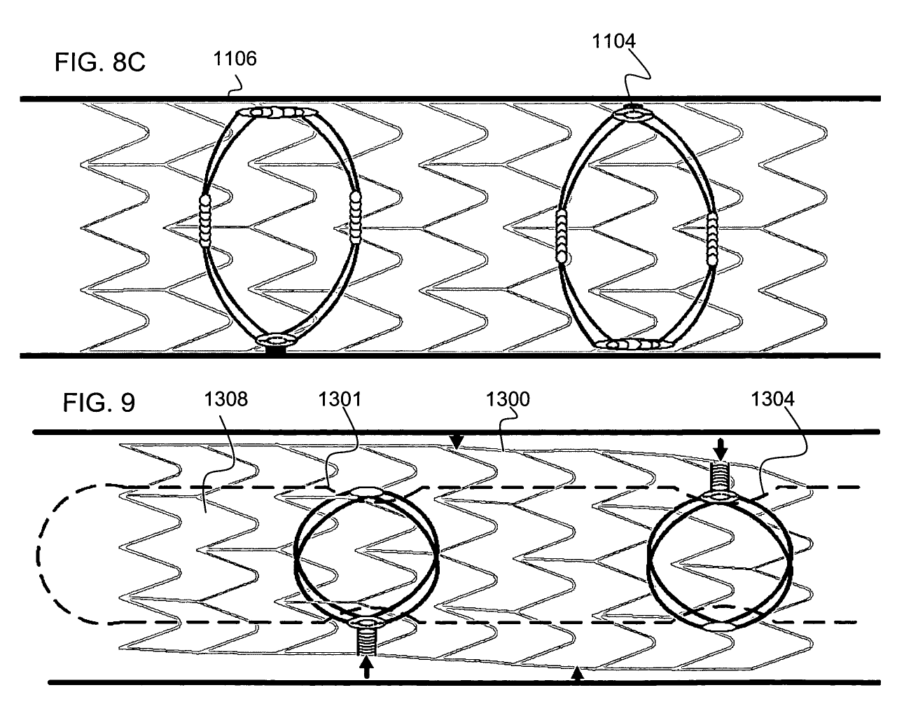

FIG. 9 depicts an alternative optional method of optionally realigning a shifted or misaligned vessel support structure 1300, comprising a rotating portion 1304 in accordance with any one of the preferred embodiments of the present invention. Vessel support structure 1300 is preferably realigned by using a grasping device 1301, optionally including but not limited to a hook. Grasping device 1301 is preferably associated with a balloon 1308 (for example from an angioplasty catheter), used to preferably realign or optionally remove support structure 1300. By partially inflating balloon 1308 and positioning the semi-inflated balloon 1308 at the center of the blood vessel lumen, the device profile decreases enabling the optional realignment or removal of vessel support structure 1300. Other grasping devices may be used additionally or alternatively.

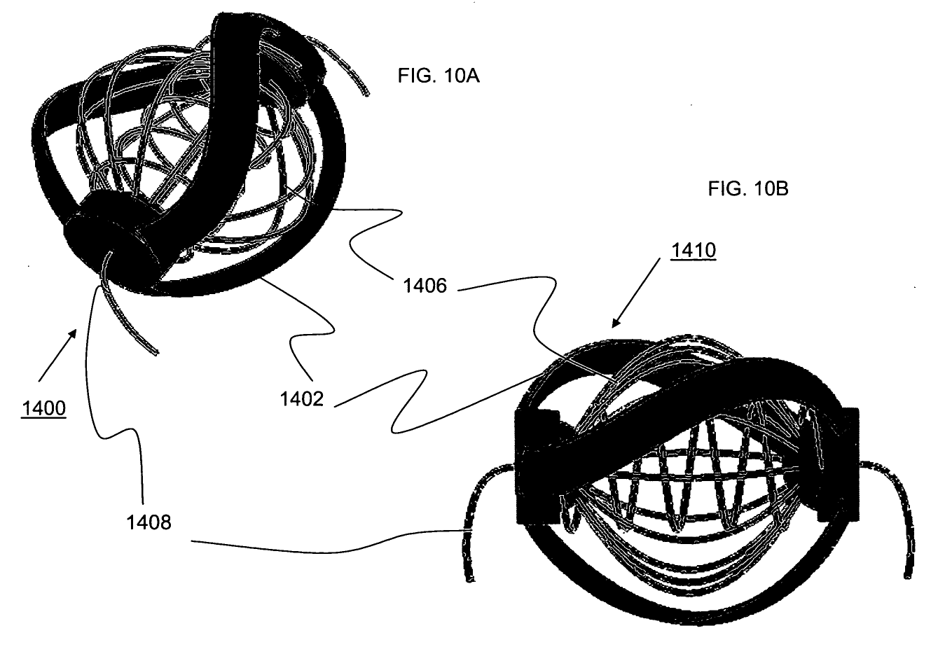

FIGS. 10A and 10B depict two planar views of the rotating portion of the device according to the present invention. Rotational devices 1400 and 1410 each preferably comprise blades 1402; wire mesh structure 1406; and anchor structure 1408. Anchor structure 1408 optionally and preferably may be coupled to various devices including but not limited to a vessel support structure (not shown), a catheter (not shown), guidewire (not shown) or the like. For example a vessel support structure (not shown) may be optionally coupled to rotating portion 1400 or 1410 in an extended or long term care application where a vessel support structure is implanted within a vessel. Optionally rotating portion 1400 or 1410 during a short term treatment may be applied, for example during surgery where rotating portion 1400 or 1410 is maintained over a cardiovascular vessel optionally by coupling it to a guidewire (not shown) or vessel support structure (not shown), optionally extending from anchor structure 1408 for the duration of the procedure. Optionally wire mesh structure 1406 may undertake various functions for example including but not limited to a filter that traps particles in the flow, and/or optionally as windings that interact with blades 1402 which are optionally coated with a magnetic substance to preferably generate electric charge. The rotation of rotating portions 1400 and 1410 may optionally also be controlled, produced, enhanced and accelerated externally by induction from an non-invasive external induction source for example including but not limited to a magnetic energy source, or by placing one or more endoluminal electrical cables that are connected to the conductive windings incorporated within a vessel support structure. Thus, accelerating the rotation of the rotating portion is optionally and preferably achieved in a similar manner as an electrical engine.

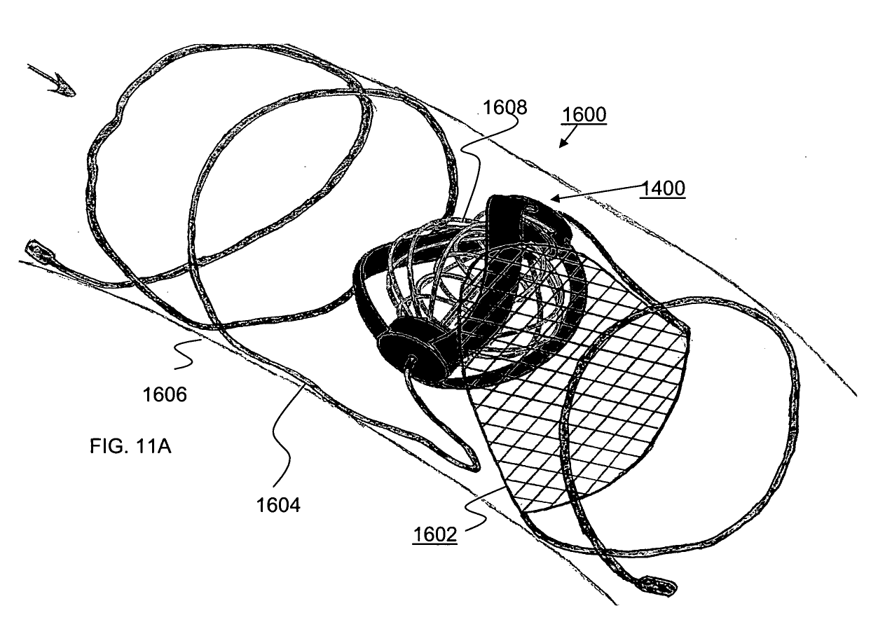

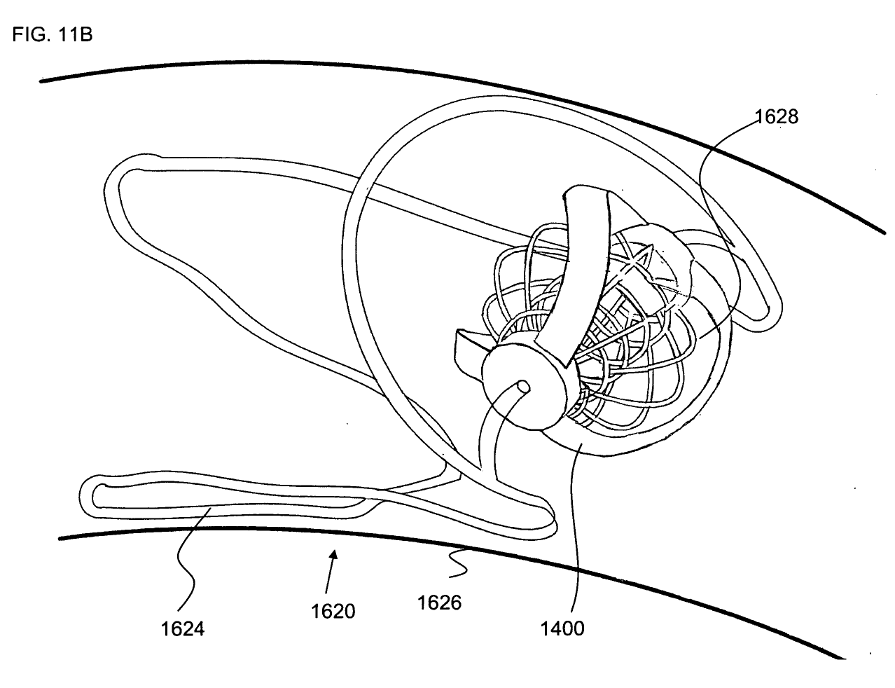

FIG. 11A depicts an exemplary embodiment of the present invention wherein vessel support structure 1600 comprises rotating portion 1400, optionally and preferably incorporating an internal filtering portion 1608 as depicted in FIG. 10A-B (as wire mesh structure 1406). A second filter portion 1602 and support structure 1604, adhering to vessel walls 1606, are also preferably provided. Optionally filter portion 1602 serves as a second stage passive filter for emboli, thrombi, or the like, where optionally rotating portion 1400 preferably serves as a first stage active filter that acts to capture and disassemble any emboli, thrombi, clots or the like, while preventing new occluding and thrombic material from forming in the vicinity of the device by any of the optional means disclosed either physically or by use of a medicament. It should be noted that the order of the second filter portion 1602 and the rotating portion 1400 relative to each other may optionally be reversed.

FIG. 11B depicts an alternative vessel support structure 1624 having a three wing configuration or tripod configuration 1620 within vessel 1626. Vessel support structure 1624 comprises rotating portion 1400 as depicted in FIGS. 10A-B, optionally and preferably incorporation an internal filtering portion 1628 as described with regard to FIG. 11A.

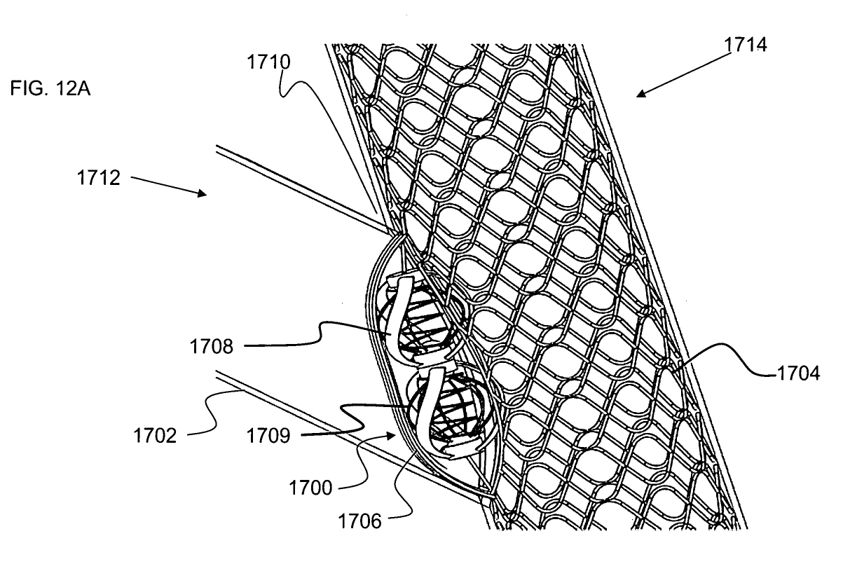

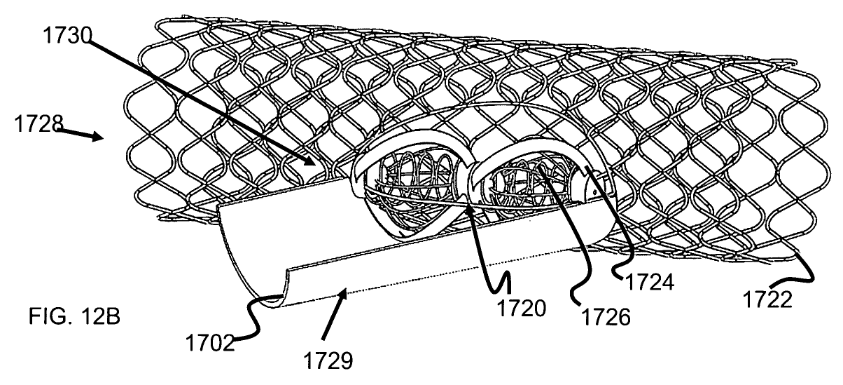

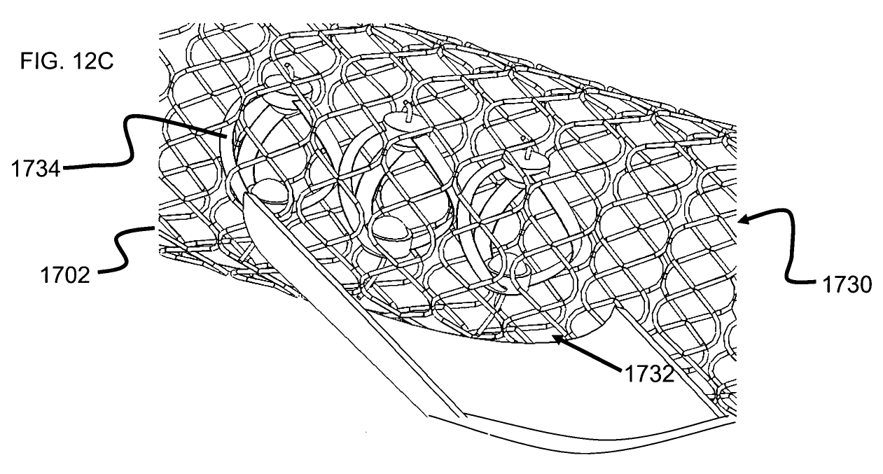

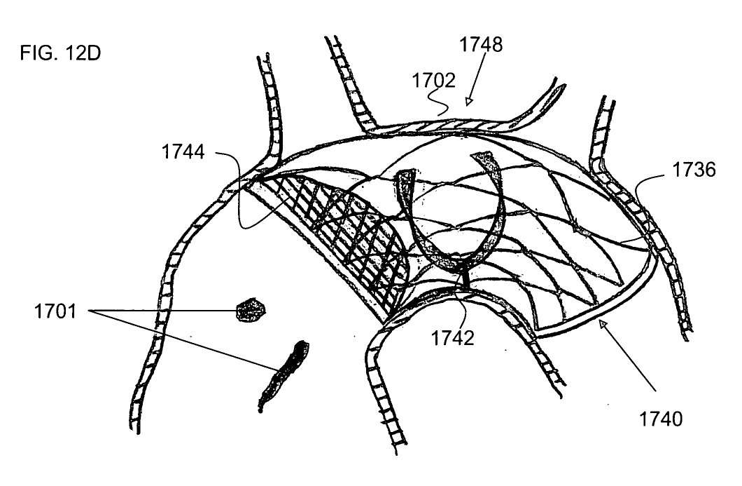

FIGS. 12A-D depict various views and positions of vessel support structure 1700 of the present invention implemented within a bifurcated vessel 1702 having bifurcation junction 1710. FIG. 12A depicts an optional formation of vessel support structure 1700 optionally comprising support structure 1704, filter potion 1706 and rotating portion 1708 within bifurcated vessel 1702. Rotating portion 1708 is optionally placed in bifurcation junction 1710 therefore optionally protecting the passageways to the ICA artery between the proximal side 1712 and the distal side 1714 of junction 1710. Rotating portion 1708 optionally lies in junction 1710 while filter portion 1706 optionally lies on distal side 1714. Optionally, filter 1706 may be placed in either the distal 1714 or proximal sides 1712 of junction 1710. Optionally and preferably rotation portion 1708 optionally comprises an internal filter 1709 which also serves as a second stage active filter that preferably disassembles occluding and thrombic material that may have formed on the device; at the same time preventing any new material from reforming. Optionally, rotation portion 1708 may generate an electric charge in order to further prevent blood components from collecting, for example optionally using windings (not shown) or using metals and/or polymers that are naturally charged in the structure, or by incorporating piezo-electric materials which may generate electric potential that generate electric charge by the rotating action.

FIG. 12B depicts an alternative and optional embodiment of a vessel support structure at bifurcate vessel 1702 which differs from that shown in FIG. 12A by not including a filter portion 1706. Bifurcate junction 1730 optionally comprises rotation portion 1724 lies in between both the distal side 1728 and proximal side 1729 of junction 1730. Optionally a filter may be implemented from within rotational portion 1724 by way of an internal filter 1726. Optionally rotation portion 1724 may be optionally used to generate and electric current as described in other embodiments of the present invention.

FIG. 12C depicts a still further alternative and optional embodiment of a vessel support structure at bifurcate vessel 1702. Vessel support structure 1730 optionally fully lies on one side of the bifurcate junction 1732, wherein the rotation portion 1734 is placed at either side of junction 1732; most preferably rotating portion 1734 may be placed on both sides of junction 1732 but does not lie within it. Optionally and preferably support structure 1730 also acts as a mesh like filter.

FIG. 12D depicts a still further alternative and optional embodiment of a vessel support structure at bifurcate vessel 1702. Vessel support structure 1740 optionally fully lies on one side of the bifurcate junction 1748, wherein vessel support structure 1740 preferably comprises rotating portion 1742, support structure 1736 and filter portion 1744. Filter portion 1744 optionally and preferably is placed to protect junction 1748 and all its outlets from entering emboli 1701. The rotating portion 1734 preferably prevents the formation of thrombi and occluding material in the vicinity of the filter portion 1744 and the bifurcate junction 1748 and all its outlets.

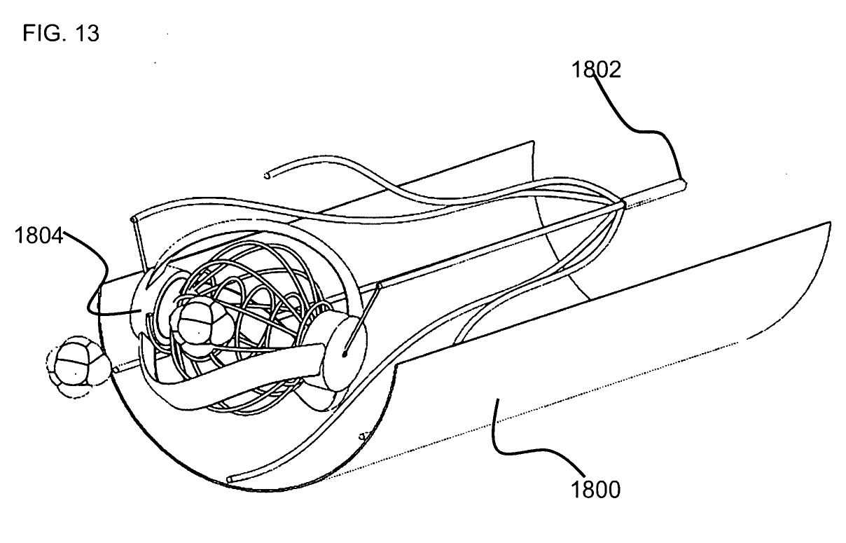

FIG. 13 depicts the double stage IVC filter 1800 wherein the rotating portion 1804 of the present invention, shown for example in FIG. 10, acts as an active filter that is optionally coupled to secondary device 1802 acting as a passive filter optionally including but not limited to an off the shelf (OTS) filter for example including but not limited to a Greenfield filter.

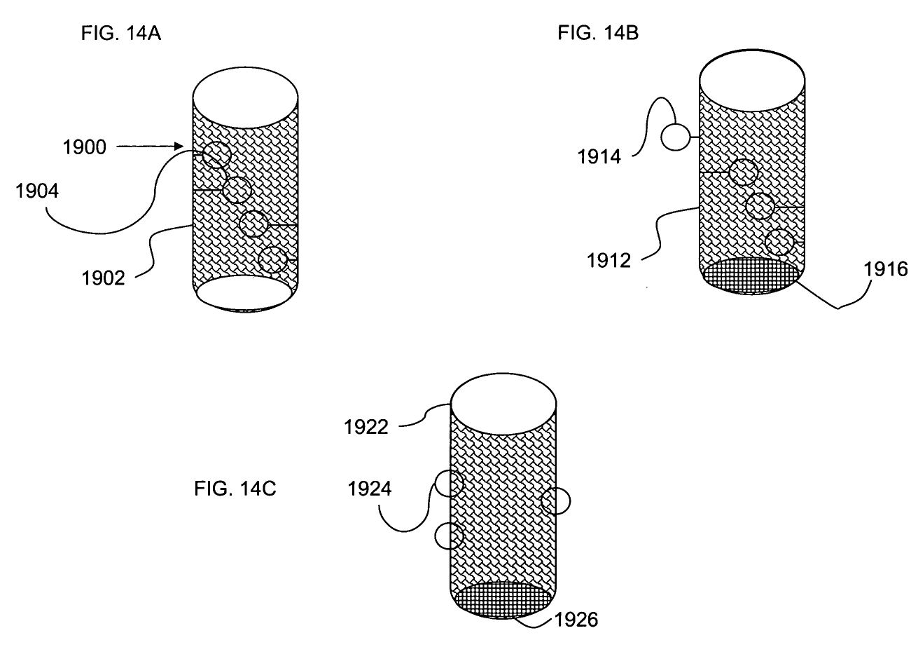

FIG. 14A depicts an exemplary device 1900 according to the present invention comprising vessel support structure 1902 and a plurality of rotating portions 1904 which are preferably placed in a stepwise and sequential manner. Optionally the plurality of rotating portions 1904 spans the inner diameter of vessel support structure 1902. Preferably the use of a vessel support structure 1900 provides an open corridor for blood flow while limiting the level of restenosis to a predefined level. Therefore, even in a situation where vessel support structure 1902 is experiencing restenosis, a passageway or corridor is preferably maintained through which fluids may flow.

FIG. 14B depicts an exemplary embodiment of a vessel support structure 1912, preferably comprising at least one rotating portion 1914, at least one filter portion 1916. Optionally rotating portion 1904 may be placed in a bifurcation junction outside the lumen of vessel support structure 1912 along its outer surface of junction.

FIG. 14C depicts an exemplary embodiment of a vessel support structure 1922, preferably comprising at least one rotating portion 1924 and at least one filter portion 1926. Optionally rotating portion 1924 may be placed along the plane of vessel support structure 1922 integrated within the vessel support structure.

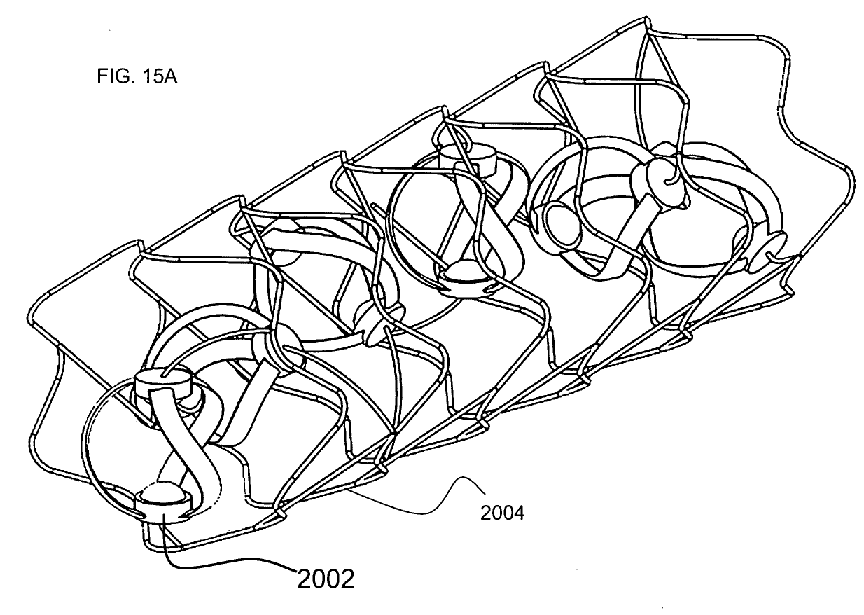

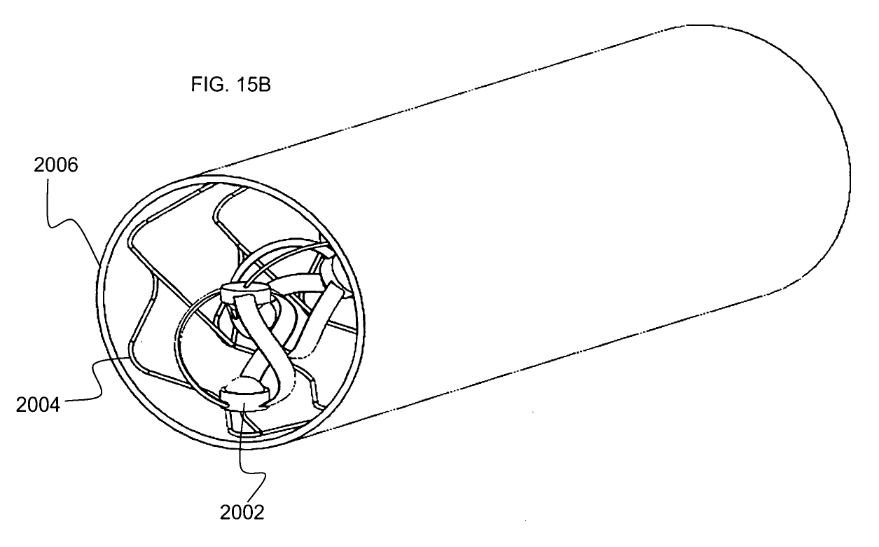

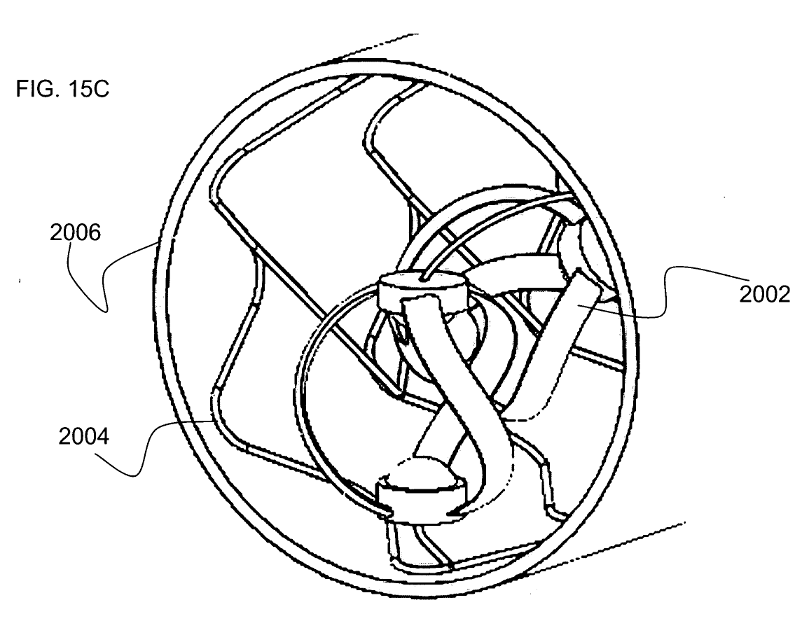

FIG. 15A provide a more detailed depiction of the embodiment of FIG. 14A, showing an optional embodiment according to the present invention wherein a plurality of rotating portions 2002 are placed sequentially and incrementally within a vessel support structure 2004 in a step like manner, to diagonally span the diameter of the vessel support structure 2004, optionally and preferably providing a corridor that limits the restenosis within the support structure's lumen. FIG. 15B is a depiction of FIG. 15A as implemented within vessel 2006. Similarly, FIG. 15C provides a perspective view of FIG. 15B.

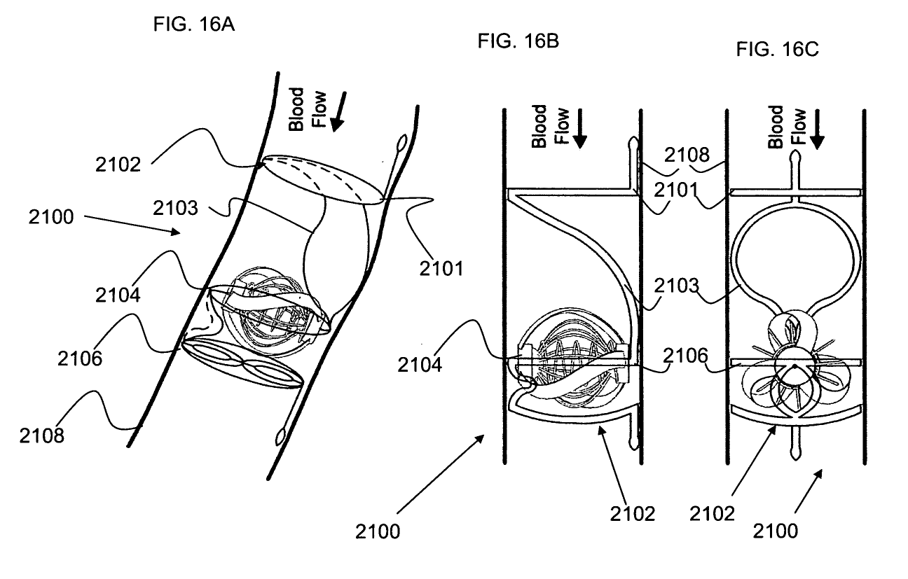

FIG. 16A-C depict an optional embodiment of filter device 2100 according to the present invention as implemented within vessel 2108, preferably comprising filter portion 2106, rotating portion 2104, and vessel support structure 2102. FIG. 16A provides a perspective view of device 2100 while FIG. 16B provides a side view and FIG. 16C provides a top view of device 2100. The vessel support structure 2102 is comprised of rings 2101 and connecting wire frame 2103 that optionally expand against the inner walls of vessel 2108, adopting the profile of the vessel inner walls 2108 by exerting a certain amount of pressure in order to retain their position; preferably at least one connecting wire 2103 extends from the rings 2101 to preferably maintain rings 2101 axially spaced from each other. The rotating portion 2104 is preferably incorporated within one of the rings 2101 in order to distance the rotating portion 2104 from the vessel walls 2108. The filter portion 2106 is also preferably incorporated within one of the rings 2101 and preferably comprises a plurality of elongated strands arranged to form a general filtering structure. Optionally filter portions 2106 and vessel support structure 2102 may take various forms as shown for example in FIG. 18.

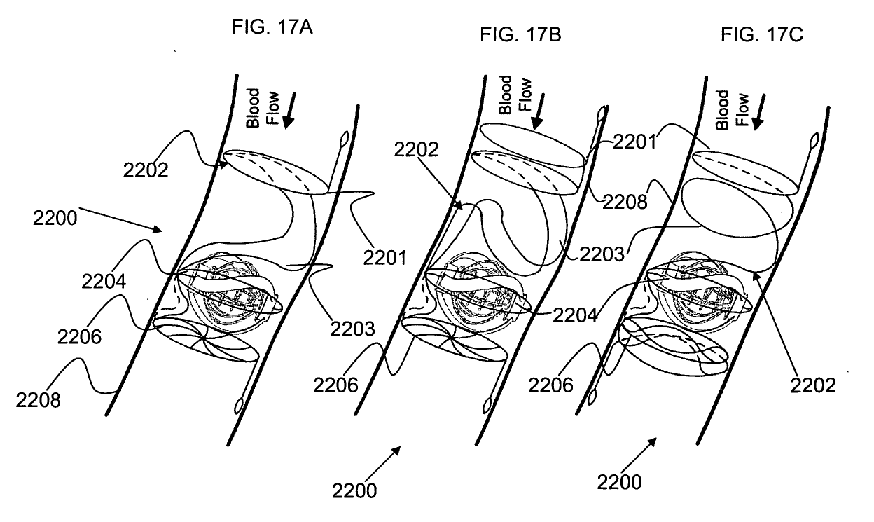

FIGS. 17A-C depict device 2200 in an optional embodiment according to the present invention as implemented within vessel 2208, preferably comprising filter portion 2206, rotating portion 2204, and vessel support structure 2202, more preferably comprising rings 2201 and connecting wires 2203 that preferably expands to fit vessel 2208. FIGS. 17A-C provides perspective views of various forms of device 2200. Optionally, filter portion 2206 may take different forms as shown in FIG. 18. Optionally vessel support structure 2202 may take different forms including different deployment of the connecting wires in order to improve structural integrity and easy insertion into a vessel.

FIG. 18A depicts an exemplary vessel support structure ring. Preferably the ring form resembles a pair of open arches that exhibit a roughly circular form in planar view (vessel profile) while having an arch-like shape when considered from the side view.

FIGS. 18B-C depict ringed vessel support structures optionally housing a rotating portion according to optional embodiments of the present invention.

FIGS. 18D-E depict ringed vessel support structures optionally housing a filter portion according to optional embodiments of the present invention.

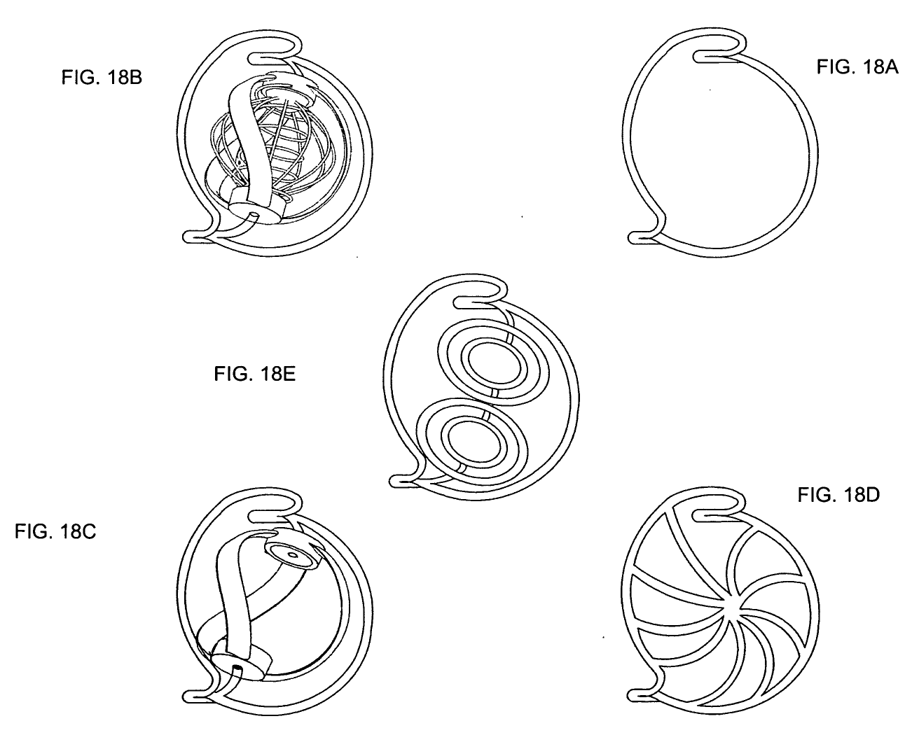

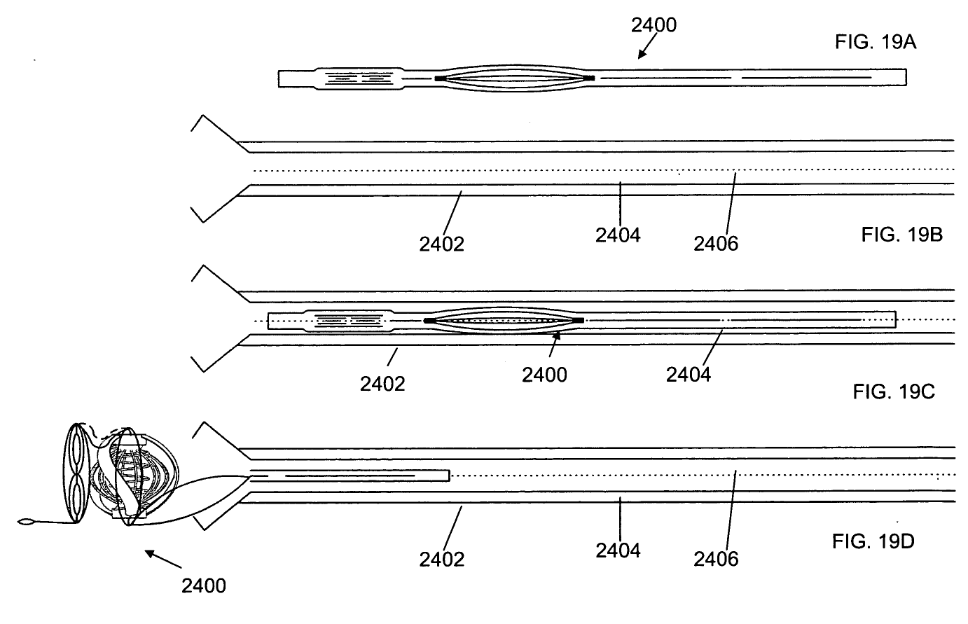

FIG. 19A-D depicts an optional delivery method according to the present invention. FIG. 19A depicts a device 2400 according to an optional embodiment of the present invention wherein the device is packaged in its collapsed linear form within a housing catheter in order to assume a delivery state, wherein all of the device components are folded onto itself and the platform is straightened to allow delivery through a minimally invasive delivery system.

FIG. 19B depicts the catheter, comprising the catheter body 2402, sheath 2404 which is preferably used to house device 2400, and guidewire 2406 used to deliver and guide device 2400 to the appropriate location. FIG. 19C depicts the loaded catheter ready for delivery and comprising FIG. 19A within FIG. 19B. The delivery catheter and device 2400 are placed within sheath 2404 loading catheter 2402 and ready for deployment a vessel. Optionally device 2400 may be fabricated in linear form mostly from a single piece of material so as to not compromise the material properties and strength while reducing the number pieces joined together.

FIG. 19D depicts the exemplary delivery of device 2400, as device 2400 is extracted from its housing it begins to unfold from its linear disposition within the delivery catheter and expand to fill the targeted area. Once device 2400 is delivered, the delivery tools catheter 2402 is preferably removed, leaving the expanded functioning device 2400.

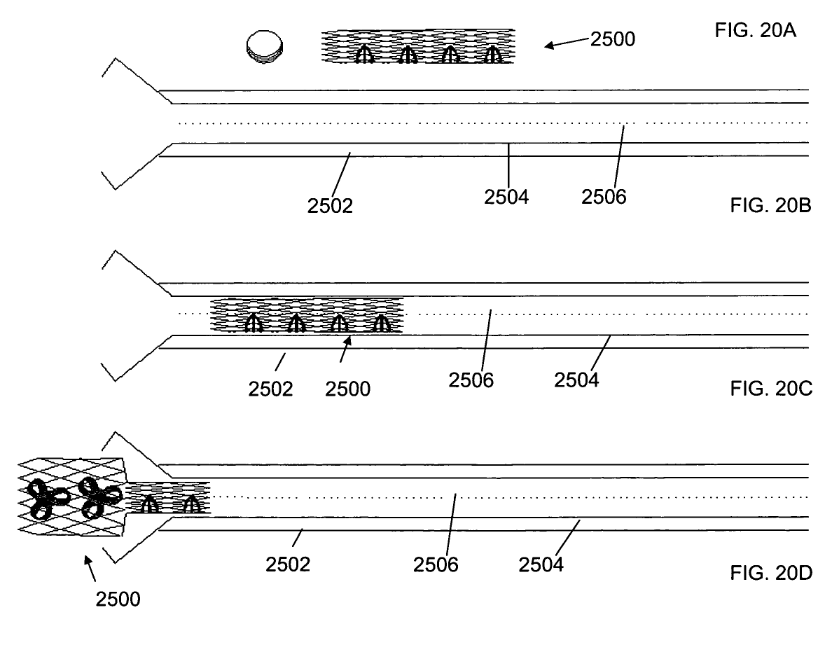

FIG. 20A-D depicts an optional delivery method according to the present invention. FIG. 20A depicts a device 2500 according to an optional embodiment of the present invention wherein the device is packaged in its collapsed linear form within a housing catheter in order to assume a delivery state, wherein all of the device components are folded onto itself and the platform is straightened to allow delivery through a minimally invasive delivery system. FIG. 20B depicts the catheter, comprising the catheter body 2502, sheath 2504 which is preferably used to house device 2500, and guidewire 2506 that is used to deliver and guide device 2500 to the appropriate location. FIG. 20C depicts the loaded catheter ready for delivery and comprising FIG. 20A within FIG. 20B. The delivery catheter and device 2500 are preferably placed within sheath 2504 loading catheter 2502 and are ready for deployment to a vessel. Optionally device 2500 may be fabricated in linear form, for example mainly from a single piece of material so as to not compromise the material properties and strength while reducing the number of pieces joined together.

FIG. 20D depicts the exemplary delivery of device 2500, as device 2500 is extracted from its housing it begins to unfold from its linear disposition within the delivery catheter and expand to fill the targeted area. Once device 2500 is delivered, the delivery tools catheter 2502 is removed, leaving the expanded functioning device 2500.

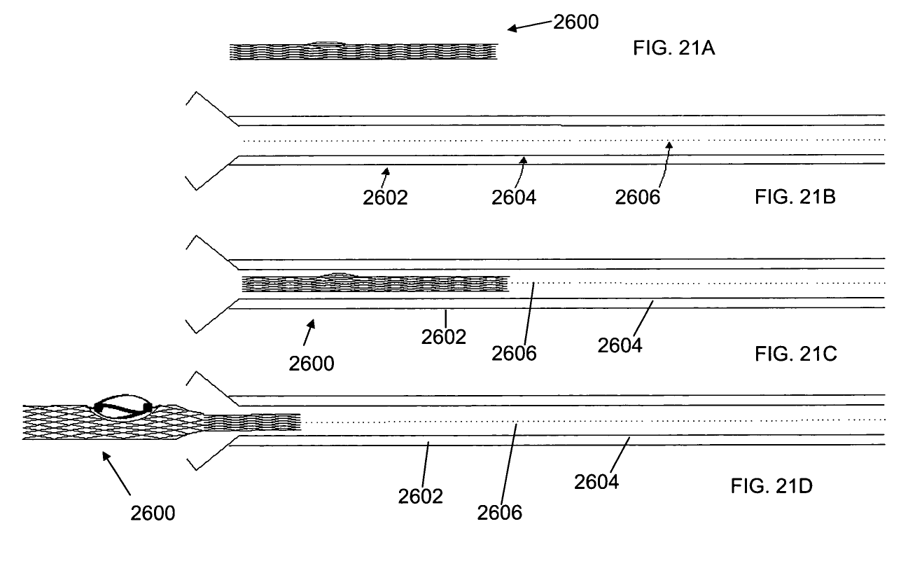

FIG. 21A-D depicts an optional delivery method according to the present invention. FIG. 21A depicts a device 2600 according to an optional embodiment of the present invention, wherein the device is packaged in its collapsed linear form within a housing catheter in order to assume a delivery state, wherein all of the device components are folded onto itself and the platform is straightened to allow delivery through a minimally invasive delivery system. FIG. 21B depicts the catheter, comprising the catheter body 2602, sheath 2604 which is preferably used to house device 2600, and guidewire 2606 that is used to deliver and guide device 2600 to the appropriate location. FIG. 21C depicts the loaded catheter ready for delivery and comprising the embodiment of FIG. 21A within the embodiment of FIG. 21B. The delivery catheter and device 2600 are placed within sheath 2604, loading catheter 2602 and thereby being ready for deployment a vessel. Optionally device 2600 may be fabricated in linear form, for example mainly from a single piece of material so as to not compromise the material properties and strength while reducing the number pieces joined together.

FIG. 21D depicts the delivery of device 2600, as device 2600 is extracted from its housing it begins to unfold from its linear disposition within the delivery catheter and expand to fill the targeted area. Once device 2600 is delivered, the delivery tools catheter 2602 is removed, leaving the expanded functioning device 2600.

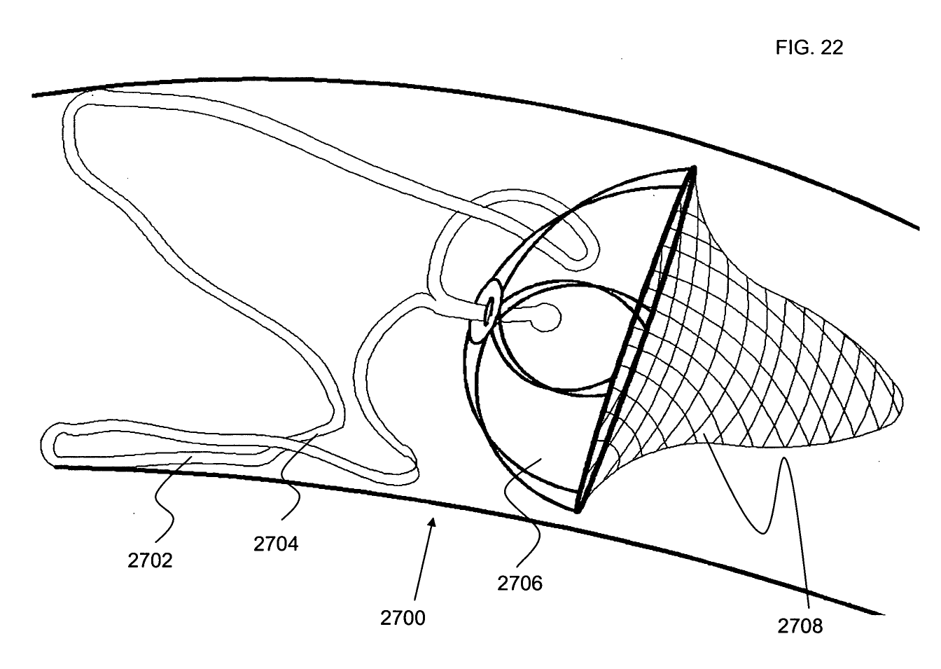

FIG. 22 shows an optional embodiment of filter device 2700 according to the present invention as implemented within vessel 2702, comprising filter portion 2708, rotating portion 2706, and vessel support structure 2704. The blood clot filtering portion 2708 is coupled to the rotating portion blades 2706 and preferably rotates together with rotating portion 2706. Rotating portion 2706 is optionally an axial flow skewback propeller rotating portion. Optionally the filter portion 2708 spans the inner diameter of vessel 2702 or part of vessel 2702 diameter. Vessel support structure 2704 is optionally implemented as a winged wire frame. Vessel support structure 2704 may take any form for example including but not limited to a stent or a filter structure. The outer wire frame typically contacts the surrounding blood vessel at one or more points along the outer wire frame.

While the invention has been described with respect to a limited number of embodiments, it will be appreciated that many variations, modifications and other applications of the invention may be made.

1. An implantable device for implanting in a blood vessel comprising a support structure, a movable blade supported by said support structure and at least one anchor for stably anchoring the support structure to a blood vessel, wherein blood flows through said movable blade.

2. The implantable device of claim I comprising a plurality of blades.

3-4. (canceled)

5. The implantable device of claim 2 comprising a plurality of blades in a concentric arrangement.

6. The implantable device of claim 1 wherein said blades have variable width along the blades structure.

7. The implantable device of claim 1 wherein said blades are helical or skewback airfoils waped into a spherical shape.

8. The implantable device of claim 7 wherein said blades shaped to a form chosen from the group consisting an elliptic shape, spherical shape, tubular shape.

9. The implantable device of claim 1 wherein said plurality of blades having a configuration chosen from the group consisting of open configuration or closed configuration.

10. (canceled)

11. The implantable device of claim 1 wherein said plurality of blades further comprise a material having a magnetic property.

12. (canceled)

13. The implantable device of claim 1 wherein said at least one anchor comprises a plurality of anchors.

14-16. (canceled)

17. The implantable device of claim 1 wherein said support structure comprises a vessel support structure and wherein said at least one rotating portion lies within said vessel support structure.

18. The implantable device of claim 1, further comprising a filter portion supported by said support structure.

19. The implantable device of claim 18 wherein said filter is within a rotational volume of said at least one blade,

20-21. (canceled)

22. The implantable device of claim 19 wherein said filter is coupled to said blades.

23. The implantable device of claim 19 wherein said filter is not coupled to said blades.

24-25. (canceled)

26. An implantable device for being implanted to a vessel having a fluid flow, comprising a vessel support structure and at least one rotating portion supported by said vessel support structure, wherein said vessel support structure comprises a lumen and wherein the fluid flows through said at least one rotating portion.

27. The implantable device of claim 26 further comprising a plurality of rotating portions.

28. The implantable device of claim 26 wherein said vessel support structure is selected from the group consisting of a tubular structure, stent, a wire frame, a stent graft, ring structure, tripod structure and a three winged structure.

29. The implantable device of claim 26 wherein said at least one rotating portion is within the lumen of the vessel support structure.

30-31. (canceled)

32. The implantable device of claim 27 wherein said plurality of rotating portions are coupled to said vessel support structure within said lumen in a sequential configuration.

33-37. (canceled)

38. An implantable device for being implanted to a vessel having a fluid flow, comprising a vessel support structure, at least one rotating portion, and a filter portion, wherein said at least one rotating portion and said filter portion are supported by said vessel support structure and wherein the fluid flows through said at least one rotating portion and said filter portion.

39. The implantable device of claim 38 wherein said at least one filter portion has a shape chosen from the group consisting of concave or convex.

40-42. (canceled)

43. The implantable device of claim 38 wherein said filter portion is attached to said rotating portion comprises a plurality of blades wherein blade configuration is chosen from the group consisting of a closed configuration or an open configuration.

44-48. (canceled)

49. The implantable device of claim 1 further comprising at least one peripheral device.

50. (canceled)

51. An implantable rotation portion for use in a blood vessel comprising a support structure, a movable blade supported by said support structure, at least one anchor for stably anchoring the support structure to a blood vessel and a medicament release mechanism connected to said support structure for releasing a medicament, wherein blood flows through said movable blade.

52. The device of claim 51 wherein the medicament is taken from the group comprising blood thinning drugs or hormones or genes.

53. (canceled)

54. The device of claim 51 wherein the medicament is controllably released.

55-57. (canceled)

58. The device of claim 52 wherein the medicament is the form of coated material.

59-67. (canceled)

68. The device of claim 1 produced from a material selected from the group consisting of bioderadable material, metal, plastic, nitinol, titanium, plastically deformable material, temperature-sensitive shape memory material with a transition temperature around body temperature, flexible thread, surgical monofilament sutures, and any elastic material.

69-70. (canceled)

71. The device of claim 38 produced from a material selected from the group consisting of biodegradable material, metal, plastic, nitinol, titanium, plastically deformable material, temperature-sensitive shape memory material with a transition temperature around body temperature, flexible thread, surgical monofilament sutures, and any elastic material.

72-73. (canceled)

74. The device of claim 26 produced from a material selected from the group consisting of biodegradable material, metal, plastic, nitinol, titanium, plastically deformable material, temperature-sensitive shape memory material with a transition temperature around body temperature, flexible thread, surgical monofilament sutures, and any elastic material.

75. A method of treating occluding matter within a blood vessel, comprising implanting a device comprising a movable blade within the blood vessel; permitting blood to flow through said movable blade; and moving said movable blade by said blood flow, wherein a movement of said movable blade breaks up the occluding matter.

76. The method of claim 75, wherein the occluding matter is selected from the group consisting of an embolus, a clot, a plaque, and any occluding material.

77-78. (canceled)

79. The method of claim 75, wherein said device further comprises a filter for filtering the occluding matter from said blood flow.

80. (canceled)

81. The device of claim 49 wherein said peripheral device is coupled to a device portion chosen from the group consisting of support structure, rotating portion, blade, anchor.

82. (canceled)

83. The device of claim 26 further comprising a peripheral device.

84. The device of claim 83 wherein said peripheral device is coupled to a device portion chosen from the group consisting of vessel support structure, rotating portion, blade.

85. The device of claim 38 further comprising a peripheral device.

86. The device of claim 85 wherein said peripheral device is coupled to a device portion chosen from the group consisting of vessel support structure, rotating portions blade, filter portion.

87. A method for implanting the device of claim 1, comprising providing the device of claim 1 in a collapsible linear form wherein the device components are folded onto themselves to allow delivery through a minimally invasive delivery system; and implanting the device with said minimally invasive delivery system.

88-92. (canceled)

93. The device of claim 1 further comprising windings and endoluminal electrical cables for being electrically connected to said at least one movable blade.

94-95. (canceled)

96. A method for determining at least one cardiac parameter, comprising: implanting a device comprising a movable blade within the blood vessel, permitting blood to flow through said movable blade; moving said movable blade by said blood flow; and determining the at least one cardiac parameter according to said moving of said movable blade.

97. The method of claim 96 wherein said cardiac parameters are selected from the group consisting of blood flow rate, cardiac output, vessel occlusion, and blood pressure.

98. The method of claim 96 wherein said cardiac parameters are determined by a sensor for sensing said blood flow.

99-115. (canceled)

116. The device of claim 26, wherein an internal surface of the vessel support structure is shaped as an airfoil for increasing blood flow through the rotating portion and farther deflecting embolic material into the rotating portion and filter portion.

117. (canceled)

118. The method of claim 87, wherein the device is retrievable.

119. (canceled)

120. (canceled)

121. The device of claim 1, wherein said support structure and said at least one movable blade arc configured to permit passage of a medical instrument.

122-124. (canceled)

125. The implantable device of claim 1, further comprising an induction device for further inducing rotation of said at least one movable blade.

126-128. (canceled)

129. The implantable device of claim 38 wherein said rotating portion comprises a plurality of airfoil blades.

130. The implantable device of claim 38 wherein said rotating portion is chosen from the group consisting of an axial-flow rotating portion or an across-flow rotating portion.
